(19) United States
(12) Patent Application Publication
(10) Pub. No.: US 2025/0208127 A1
(43) Pub. Date: Jun. 26, 2025

(54) MICELLAR MYCOLATE COATED CARBON ELECTRODES FOR ELECTROCHEMICAL IMPEDANCE IMMUNOASSAY

(71) Applicant: University of Pretoria, Pretoria (ZA)

(72) Inventors: Jan Adrianus VERSCHOOR, Pretoria (ZA); Arthessa RAGAVALOO, Pretoria (ZA); Mosa Jennifer MOLATSELI, Pretoria (ZA); Carl Robert BAUMEISTER, Pretoria (ZA); Ikechukwu Emmanuel OKEKE, Pretoria (ZA)

(73) Assignee: University of Pretoria, Pretoria (ZA)

(21) Appl. No.: 19/073,632

(22) Filed: Mar. 7, 2025

Related U.S. Application Data
(63) Continuation of application No. PCT/IB2023/058996, filed on Sep. 11, 2023.

(30) Foreign Application Priority Data
Sep. 12, 2022 (ZA) 2022/10098

(51) Int. Cl. G01N 33/543; G01N 27/02; G01N 33/569

(52) U.S. Cl. CPC G01N 33/5438; G01N 27/026; G01N 33/5695; G01N 2333/35; G01N 2469/20

(57) ABSTRACT

A method of forming a solution of mycolic acid antigens for immobilisation on a substrate is provided. The method comprises heating a mixture of mycolic acid and a polar organic solvent to a temperature higher than the melting point of the mycolic acid, thus producing a solution of mycolic acid antigens in the polar solvent, wherein the solution is a micellar solution.

### CROSS REFERENCE TO RELATED APPLICATIONS

This continuation application claims priority benefit from International Application No. PCT/IB2023/058996 filed on Sep. 11, 2023, which claimed priority from South African Application No. 2022/10098 filed Sep. 12, 2022, which are both incorporated herein by reference in their entirety.

### FIELD OF THE INVENTION

This invention lies in the field of medical devices for disease diagnostics and methods of using such devices for the detection of TB biomarker antibodies to antigens in human and animal blood and tissue samples. In particular, the invention relates to an improved method of preparing substrates, such as electrodes, coated with immobilised mycolic acid antigens for use in impedimetric detection of mycolic acid biomarker antibodies, wherein the mycolic acid antigens are presented as biomarker antibody capture agents.

### BACKGROUND OF THE INVENTION

### The Global Need for Diagnosis of TB

Tuberculosis (TB), although curable, remains a leading cause of death by infectious disease worldwide. It is caused by Mycobacterium tuberculosis (Mtb). The latest statistics released by the World Health Organization (WHO) in 2021 showed more than 1.5 million TB deaths, of which approximately 1.3 million were among HIV-negative people and 0.2 million among HIV-positive people. It is estimated that globally, 1.7 billion people live with latent TB (asymptomatically infected with Mtb) of which approximately 10% are likely to develop active (symptomatic) TB within 2 years after initial exposure. The risk of reactivation of latent TB is remarkably high among individuals infected with HIV.

The WHO aims to reduce TB-related mortality by 95% and incidence by 90% between 2015 and 2035 through its End TB strategy. This goal is to be achieved with more effective vaccines, treatment regimens and improved diagnostic tests.

Current diagnostic tools in routine clinical use today rely mostly on sputum-based testing, which has consistently demonstrated poor sensitivity, especially in immune-compromised individuals and children who are unable to produce sputum of the desired consistency. There have been tremendous improvements in diagnostic tests for TB over the last few years. Despite these advances, no test can meet all the required specifications in terms of performance (sensitivity and specificity), ease of use, cost, rapidity of diagnosis, and the ability to generate same-day results at the point of care (POC). The most important contribution to the End TB strategy will be POC platforms which will make TB diagnosis more accurate, affordable and widely available for patients and care providers. Despite existing technologies and advances over the last few decades, development of a simple and rapid POC test remains a challenge in the current pipeline of tuberculosis diagnostics. For this purpose, novel diagnostic tools and reliable biomarkers in samples other than sputum are required.

### Advantage of a Blood-Based TB Diagnostic

Blood is an attractive sampling option for the detection of TB, especially in HIV-infected patients. Blood is easier and more reliable to sample than sputum because the invasiveness of sampling is negligible, requiring just a finger prick of blood. Sputum sampling is also inadequate to diagnose extrapulmonary TB, TB in children and TB in HIV-co-infected individuals. Blood sampling is better able to overcome these limitations. Many serological tests have been proposed in the past for TB diagnosis. Serological tests include enzyme-linked immunosorbent assay (ELISA) or lateral flow immunoassays (LFIA) to detect the humoral antibody response to Mtb antigens by measurement of antigen-antibody interactions. Serological tests have the potential to be suitable and improve diagnosis in resource-limited areas because they are simple and offer the potential of low cost, rapid diagnosis with minimal training requirements.

### Antibody Biomarkers in TB Diagnosis

Antibodies or immunoglobulins are proteins produced by B cells that defend a host against foreign agents such as viruses and bacteria. Antibody-based immunoassays are commonly used in diagnostics. They are based on the avidity and specificity of antibodies to bind and recognise pathogen-related antigens. Immunoglobulin G (IgG) is the major blood antibody of the secondary immune response associated with prolonged immunity whereas immunoglobulin M (IgM) is the predominant early blood antibody seen in the immune response to infectious diseases but usually with short-lived memory. Immunodiagnostic assays based on detection of patient IgM and IgG against pathogen-related antigens in patient sera with active TB disease is an attractive approach for rapid POC diagnosis or screening.

Studies towards the identification of new biomarkers for TB diagnosis have increased in recent years. A good antibody biomarker should be highly specific to a disease, easily detectable by standard antibody detection methods and able to distinguish between latent and active stages of Mtb infection. The early detection of TB is another important aim in biomarker research, in particular in patients that indicate risk of contracting or relapsing to active TB. As indicators of disease, antibodies are well suited for use in diagnostics because of their renowned properties of sensitivity and specificity. The advantage of detecting antibodies, rather than pathogens and their traces, is that antibodies are freely available in serum, whereas microorganisms can evade detection by shielding themselves within native cells in different organs of the body.

Previous work suggests that antibody production to the Mtb antigen mycolic acids (MA), the unique lipid antigens of mycobacteria, could serve as an ideal biomarker for active TB diagnosis. This is because these antibodies are produced independently of CD4 helper T-cells and so are unaffected by HIV co-infection. Furthermore, work by Ndlandla (1) using a guinea pig TB infection model indicated that anti-MA antibodies are produced early upon infection, are short-lived and will therefore diagnose active TB independently of previous vaccination and even under the condition of HIV co-infection.

### Interference by Anti-Cholesterol and Rheumatoid Factor Antibodies

While MAs are attractive antigens for use in TB diagnostic tests, cholesterol gives rise to cross-reactivity in TB detection. The packed structure of cholesterol has been shown to be analogous to that of MAs as demonstrated by the indistinguishable binding of amphotericin B (AmB)—a cholesterol binding agent—to both MAs and cholesterol. Consequently, the ubiquitously present natural anti-cholesterol IgG and IgM antibodies produced by the human body also recognise the MAs, leading to cross-reactivity that potentially gives false positive results for TB-negative patients. Moreover, some of the recombinant anti-MA single-chain variable fragments (scFvs) from the Nkuku gene library have been shown to cross-react with cholesterol (2), although the avidity of binding of recombinant chicken antibodies (gallibodies) to cholesterol was much lower than for binding MA.

Rheumatoid factors (RFs) are antibodies directed against the Fc region of IgG. They consist of different isotypes and bind fragment crystallizable regions (Fc) with different affinities. Although associated with rheumatoid arthritis, they were also found to occur in more than 60% of patients suffering from TB. By their non-specific cross-linking of IgG, rheumatoid factors can enhance the avidity of low affinity antibodies to a level that can compete in the same range as higher affinity antibodies; thereby interfering with the accuracy of immunoassays. In active TB the cross-reactivity between MA and cholesterol may therefore become problematic when patient blood also contains rheumatoid factors. Rheumatoid factors should therefore be controlled in immunoassays aimed at TB diagnosis by means of TB biomarker antibody detection.

### MA Conformation Determined by Solvent and Surface Curvature

Groenewald et al. (3) demonstrated how MA folding is influenced by the solvent in which they are dissolved, thus allowing for different conformations in different solvents. In particular, they determined that stable folded MA conformations are hindered more in hexane than in water, with the exception of keto-MA which readily formed the so-called W-conformation in hexane, which Ranchod et al. (2) suggested to be most likely associated with the cholesteroid cross-reacting conformer of MA. In addition, the size of antibody capturing particles was shown to be critical for MA conformation. Baumeister, Shaw and Verschoor (4) demonstrated that the higher curvature of smaller MA-containing liposomes made the MA fold into a conformation that was better able to capture anti-MA antibodies, implying that the conformation of MA is also influenced by the diameter of the MA-coated antibody-capturing particle onto which MA is immobilised. It is therefore important that the solvent for dissolution of MA during the manufacture of MA-coated electrodes is carefully selected, as well as the solid phase sensor surface properties of the electrodes that are to be coated with MA.

### The MARTI Assay for TB Diagnosis

Human patient anti-MA antibodies have been considered before as biomarkers for active TB diagnosis with the potential of being detectable by means that are appropriate for POC TB testing. This idea was first explored by Schleicher et al. (5), using an ELISA assay and MA as the antigen to detect anti-MA antibodies found in patient sera. The results showed that there were more anti-MA antibodies present in TB-positive patients compared to TB-negative patients. Due to this result, further experiments were conducted to produce a diagnostic test that uses MA antigen and anti-MA antibodies to detect active TB (6, 7, 8). The Mycolic acid Antibody Real Time Inhibition (MARTI) assay was developed from this concept. MARTI makes use of immobilised liposomes (carrying MAs) on sensor surfaces to monitor the binding of anti-MA antibodies. The use of biosensor-based technology for TB diagnosis has been well demonstrated in wave guide (8) and surface plasmon resonance (SPR) (9) evanescent field biosensors, while proof of concept has also been demonstrated in electrical impedance spectroscopy (EIS) (10, 11). The proposed MARTI test showed positive results for diagnosis of TB using this principle. However, it was shown that the biosensor technique is very difficult and costly to perform in the laboratory and that the above-mentioned technologies are still too advanced to be used in a POC setting for screening large numbers of patient sera.

The standard ELISA immunoassay is an ineffective TB diagnostic tool due to its inherent property of registering the binding of only the highest affinity antibodies to antigen in a serum. This is because the washing steps required in ELISA remove the low affinity antibodies. In comparison to the ELISA assay, the MARTI test has an increased sensitivity and specificity because it does not require a washing step after sample contact with the immobilised MA antigen (9, 12). A major advantage of the MARTI test is therefore that it can sensitively detect low-affinity antibodies, making it a more accurate diagnostic test.

### A Point of Care Version of the MARTI-Assay

EIS is more suitable to a POC diagnostic than SPR evanescent field biosensing as transduction technology for antibody binding detection in MARTI, since it requires no complex benchtop instrumentation. Signal processing can nowadays be done by means of a hand-held, battery-operated potentiostat. Such potentiostats are essentially service-free, unlike SPR which has moving parts that require regular maintenance. A POC TB diagnostic device must be affordable, accurate, simple to use, require minimal amounts of biological sample, be sensitive and specific, be easy to read, be able to diagnose rapidly and be able to generate same day results. With affordable, disposable electrodes, the MARTI test on EIS holds the potential to fulfil these requirements.

MA antigens have previously been immobilised from hexane solution onto solvent-resistant screen-printed gold electrodes (wherein “screen-printed electrodes” may be abbreviated as “SPEs”) coated with octadecanethiol (ODT) to provide a self-assembled monolayer on the gold that is a requirement for sensitive detection of binding ligands with EIS. This method of antigen immobilisation is, however, poorly reproducible and wasteful on MA. It is therefore necessary to identify a more reproducible and affordable protocol of MA antigen immobilisation on SPEs. In addition, a nanoparticle antibody capture agent is required in the MARTI-test, which was hitherto based on MA-containing liposomes and poly(lactic-co-glycolic acid) (PLGA) particles. Both these particle types proved to be of too limited stability to be feasible for inclusion into a POC TB diagnostic for use in the field.

### Electrode Advances: Use of Carbon Electrodes

Previously reported data indicated high variability in the MARTI assay's performance due to inconsistencies in electrode coating. A scientific publication disclosed that the hexacyanoferrate and gold combination is essentially an unstable system (13). Organic solvents (typically hexane) were previously used for MA-coating and many of these solvents are incompatible with carbon electrodes. The use of a polar organic solvent, such as acetone, for MA coating and its compatibility with carbon electrodes and the dielectric mask, as disclosed in accordance with the present invention, unexpectedly enabled the change to a screen-printed carbon electrode, as described in accordance with the invention and exemplified in the examples.

Variability is inherent when using carbon inks as part of the screen-printing process for electrode manufacture due to impurities and organic binders that are essential in carbon inks and are improperly removed causing contamination and variation during the curing process. The variability is apparent when impedance measurements for cured electrodes are compared. Impedance data typically range from 600Ω to 4000Ω. This inter-electrode variation in impedance values negatively affects the performance of MA-coated electrodes. The use of carbon electrodes, as has been enabled by the present invention, required investigation into pre-treatment methods to minimise the inter-electrode variation. Three alternative pre-treatments were investigated: a non-organic treatment at raised pH levels; immersion in an ultrasonic bath; and removal by exposure to several less aggressive organic solvents. The data showed that, without any reasonable expectation of success and, therefore, surprisingly, the third alternative was the most effective, specifically that a polar organic solvent, such as acetone, successfully pre-treated the carbon electrodes prior to coating with MA without destruction of the electrode. The exposure time of acetone was titrated to establish an effective range of operation. This pre-treatment step reduced inter-electrode variation to acceptable levels by decreasing the presence of organic impurities used in the electrode production process.

The directive of the disclosed invention includes to provide for EIS detection of TB biomarker antibodies to MA antigens in human and animal blood and tissue samples in which the SPEs can be reproducibly and affordably coated with MA.

The invention also seeks to provide, independently but also cumulatively, for the nanoparticle immunosorbent step of existing methods to be replaced by a more reliable way to avoid the interference of cross-reactive and RF antibodies.

In achieving its purpose, the invention discloses, inter alia, the novel use of a polar organic solvent to prepare mycolic acid solutions for the preparation of substrates, such as electrodes, on which mycolic acid antigens are immobilised for use in binding anti-mycolic acid antibodies in detecting active tuberculosis infection in a patient. MA from the cell wall of Mtb is not soluble in a polar solvent, such as acetone. Unexpectedly, it was found by the inventors that MA forms a micellar solution in acetone after heating and cooling. The micellar solution can then be applied to coat biosensor electrodes with MA. Furthermore, acetone is a polar solvent (unlike n-hexane which is widely used in similar technologies) and was found, advantageously and unexpectedly, to cause MA to coat biosensor electrodes by self-assembling into capacitive surfaces in the required conformations for sensitive electro-impedimetric measurements. This was found to be advantageous over earlier technologies using n-hexane, since it obviates the required step, associated with the use of n-hexane, of pre-coating SPEs, and more particularly gold SPEs, with long-chain alkane-thiols in order to provide a surface for antigen immobilisation.

More specifically, when the micellar solution in acetone is applied to the electrode surface, the MA was found unexpectedly to fold itself appropriately into the desired antigenic conformation to detect and bind with TB biomarker antibodies. It is therefore not necessary to first form a priming, hydrophobic self-assembled monolayer as required by previous technologies using n-hexane as mycolic acid solvent.

### SUMMARY OF THE INVENTION

According to a first aspect of the invention, there is provided a method of forming a solution of mycolic acid antigens, the method comprising heating a mixture of mycolic acid and a polar organic solvent to a temperature higher than the melting point of the mycolic acid, thus producing a solution of mycolic acid antigens in the polar solvent.

The solution of mycolic acid antigens in the polar solvent may, in particular, be a micellar solution.

The solution of mycolic acid antigens may be a solution for immobilisation, specifically of the mycolic acid antigens, on a substrate to bind, in use, anti-mycolic acid antibodies. Such anti-mycolic acid antibodies may typically be comprised in a blood or tissue sample of a human or animal patient having tuberculosis which may in use be contacted with the substrate having the mycolic acid antigens immobilised thereon.

Throughout this specification, the singular term “mycolic acid” should be understood also to include the plural (i.e. “mycolic acids”), since in most, if not all, instances, mycolic acid would comprise a combination of two or more different mycolic acids. This is a natural result of the biosynthesis of mycolic acid.

Furthermore, in this specification, except where it is expressly stated otherwise or the context clearly requires otherwise, the terms “mycolic acid” and “mycolic acid antigen” are used interchangeably, in the sense that mycolic acid is an antigen and, more specifically, a Mtb antigen. In this sense, the singular “mycolic acid antigen” should be understood also to include the plural (i.e. “mycolic acid antigens”), for the same reason as in the case of the singular “mycolic acid” as discussed above.

Still further, except where it is expressly stated otherwise or where the context clearly requires otherwise, reference to mycolic acid should be interpreted to include not only mycolic acid as such, but also to include mycolates that comprise mycolic acid. Therefore, it follows that the anti-mycolic acid antibodies may equally be anti-mycolate antibodies.

Mycolate is a general term for wax compounds derived from the cell envelope of tuberculous mycobacterial species. More specifically, mycolates can be any salt or ester of mycolic acids. Examples include alpha-, keto- and methoxy mycolic acids. Examples of mycolate esters include glycolipids-mono- and di sugar esters of mycolic acids including glucose monomycolate, trehalose monomycolate, trehalose dimycolate or arabinogalactan esters of mycolic acids.

The mycolic acid may, in particular, be derived from tuberculous mycobacteria, such as Mycobacterium tuberculosis, or may be a synthetic analogue of such mycolic acid.

The temperature higher than the melting point of the mycolic acid may be a temperature between 60° C. and 90° C.

The method may include cooling the solution of mycolic acid antigens in the polar solvent to a temperature between 25° C. and 35° C., wherein the polar organic solvent may be acetone.

The method may include a prior step of forming the mixture of mycolic acid and the polar organic solvent.

The step of forming the mixture of mycolic acid and the polar solvent may comprise adding mycolic acid to the polar solvent. Such addition may be to provide a concentration of between 0.05 and 0.25 mg/ml, of mycolic acid in polar organic solvent. It will be understood that such addition would need to be in an appropriate amount and volume to achieve the specified mycolic acid concentration.

The invention extends, as a second aspect thereof, to a solution of mycolic acid antigens in a polar organic solvent produced according to the method of the first aspect of the invention. Wherein the solution may be a micellar solution.

According to a third aspect of the invention, there is provided a method of immobilising mycolic acid antigens on a substrate, the method including applying the solution of mycolic acid antigens in a polar solvent according to the second aspect of the invention to a substrate on which mycolic acid antigens are to be immobilised.

The immobilisation of the mycolic acid antigens on the substrate may be to bind anti-mycolic acid antibodies in use. Such anti-mycolic acid antibodies may typically be comprised in a blood or tissue sample of a human or animal patient having tuberculosis, which may in use be contacted with the substrate having the mycolic acid antigens immobilised thereon.

The method may include a prior step of producing the solution of mycolic acid antigens in a polar solvent according to the method of the first aspect of the invention.

The step of applying the solution of mycolic acid antigens in a polar solvent to the substrate may be performed after heating of a mixture of mycolic acid and a polar organic solvent according to the method of the first aspect of the invention.

More specifically, the step of applying the solution of mycolic acid antigens in a polar solvent to the substrate may be performed after cooling of the mixture after heating.

The step of applying the solution of mycolic acid antigens in a polar solvent to the substrate may be performed, for example, within about four hours after such heating and, preferably, cooling.

Several aliquots of the solution may be applied sequentially to the substrate.

In accordance with the invention, application of the solution to the substrate causes the mycolic acid antigens to self-assemble into capacitive surfaces on the substrate in the sense that the mycolic acid folds itself appropriately into the desired antigenic conformation to detect and bind with anti-mycolic antibodies in a sample contacted with the substrate in use.

The method may include leaving or causing the solution of mycolic acid antigens in a polar solvent to dry on the substrate. The substrate is dried to remove all volatiles. The substrate may be dried under high vacuum for a period of up to 16 hours. Wherein the substrate may be free of a surface modifying monolayer.

The method may include a subsequent step of blocking non-specific binding sites on the substrate according to the method of the seventh aspect of the invention. Wherein the substrate may be an electrically conductive substrate.

The substrate may, more specifically, be an electrode. Typically, the substrate would comprise a solid surface of the electrode. Wherein the electrode may, in particular, be a screen-printed carbon electrode.

The screen-printed carbon electrode may, for example, be a screen-printed carbon electrode prepared according to the method of the fifth aspect of the invention, i.e. being a screen-printed carbon electrode in accordance with the sixth aspect of the invention.

The method may include a prior step of preparing the screen-printed carbon electrode according to the method of the fifth aspect of the invention.

Applying the solution to the substrate may comprise depositing the solution onto the substrate. Such depositing may, for example, be performed by spraying, jetting, printing, spotting, etc.

The invention extends, as a fourth aspect thereof, to a screen-printed carbon electrode comprising mycolic acid antigens immobilised thereon, produced according to the method according the third aspect of the invention.

According to a fifth aspect of the invention, there is provided a method of preparing a screen-printed carbon electrode for immobilising an antigen thereon in the absence of a surface modifying monolayer, the method including treating the electrode, on which an antigen is to be immobilised, with acetone. Wherein treating the electrode with acetone may include contacting the electrode with acetone.

Contacting the electrode with acetone may include:
submerging the electrode in, or spraying or flowing the electrode with acetone; and/or
rinsing the electrode with acetone,
   wherein, if both submerging and rinsing are performed, rinsing is performed after submerging.

The electrode may be submerged in acetone to remove some of the organic binders and impurities. This could typically take between 2 and 7 minutes. The electrode may be submerged in and/or rinsed with an excess of acetone. In the case of rinsing, for example, a volume of acetone≤0.5 ml may be used. The method may also include drying the electrode after treating the substrate with acetone.

The electrode may be dried inside an oven and allowed to cool. The electrode may, for example, be dried for between 10-30 minutes in an oven at about 80° C. and allowed to cool to room temperature for between 3-7 minutes.

The method may be performed subject to the proviso that the method omits a step of applying a surface modifying monolayer to the electrode.

The method may include performing, subsequently, the method of the third aspect of the invention. The method may also include performing, subsequently, the method of the seventh aspect of the invention.

The invention extends, as a sixth aspect thereof, to a surface modifying monolayer-free screen-printed carbon electrode prepared according to the method according to the fifth aspect of the invention.

According to a seventh aspect of the invention, there is provided a method of blocking non-specific binding sites on a substrate, the method comprising treating the substrate with a protein hydrolysate.

The protein hydrolysate may be provided as a solution thereof. The solution may, for example, comprise 0.1 to 3% w/v protein hydrolysate. Wherein the protein may be casein.

Treating the substrate with casein hydrolysate may include incubating the substrate in the protein hydrolysate or dispensing the protein hydrolysate onto the substrate more specifically in the protein hydrolysate solution.

The period of incubation of the mycolic acid coated electrodes in casein hydrolysate may range from 1-16 hours.

The substrate may, in particular, be a substate on which antigens from pathogens have been immobilised.

For example, the substrate may be a substrate on which mycolic acid antigens have been immobilised according to the method of the third aspect of the invention. The method may therefore include a prior step comprising the method of the third aspect of the invention.

The substrate may be an electrode. In one embodiment of the invention, the electrode may be a screen-printed carbon electrode.

For example, the substrate, or electrode, may be according to the fourth aspect of the invention. The method may therefore include a prior step comprising the method of the third aspect of the invention.

The invention extends, as an eighth aspect thereof, to a substrate of which non-specific binding sites have been blocked according to the method of the seventh aspect of the invention.

According to a ninth aspect of the invention is provided a method of detecting tuberculosis biomarker antibodies in a human or animal blood or tissue sample, the method including contacting a substrate on which mycolic acid antigens have been immobilised according to the method of the third aspect of the invention, or a substrate according to the fourth aspect of the invention, with a sample from a patient suspected of having active tuberculosis in order to allow any biomarker anti-mycolic acid antibodies in the sample to bind to the immobilised mycolic acid antigens.

The method may include using electrochemical impedance spectroscopy to measure the degree of antibody binding to the immobilised mycolic acid antigens in the sample, wherein any measured signal higher than a previously measured baseline signal of the same substrate is a result of biomarker anti-mycolic acid antibodies binding to the immobilised mycolic acid antigens and is indicative of active tuberculosis in the patient.

The substrate may in addition, prior to contacting with the sample, have been subjected to the method of the seventh aspect of the invention.

The substrate may therefore instead be a substrate, or electrode, according to the fourth or eighth aspects of the invention.

More specifically, according to the ninth aspect of the invention, there is provided a method of detection of TB biomarker antibodies in human and animal blood and tissue samples, the method comprising:
incubating a sample from a patient suspected of having active tuberculosis with a rheumatoid factor blocker solution, thereby producing a rheumatoid factor-blocked sample;
diluting the rheumatoid factor-blocked sample in redox buffer, thereby producing a dilution of the rheumatoid factor-blocked serum sample;
optionally, chemically blocking, preferably in accordance with the seventh aspect of the invention, non-specific antibody binding sites on a mycolic acid antigen-coated electrode prepared according to the third aspect of the invention, if the electrode has not previously been subjected to treatment to effect such chemical blocking in accordance with the method of the third aspect of the invention;
measuring an electro-impedance baseline signal of the chemically blocked, mycolic acid-coated electrode in redox buffer with a potentiostat;
contacting the dilution of the rheumatoid factor-blocked serum sample with the chemically blocked screen-printed electrode containing the immobilised mycolic acid antigens in order to allow any biomarker anti-mycolic acid antibodies in each sample to bind to the immobilised mycolic acid antigens; and
using electrochemical impedance spectroscopy to measure the degree of binding of anti-mycolic acid antibodies in the sample to the immobilised mycolic acid antigens, wherein any measured signal higher than the previously measured baseline signal of the same electrode is a result of biomarker anti-mycolic acid antibodies binding to the immobilised mycolic acid antigens and is indicative of active tuberculosis in the patient.

Anti-cholesterol IgG and IgM antibodies cross-react with MA and may produce false positive results (5). As rheumatoid factor binds non-specifically to IgG and increases the binding of low affinity antibodies, rheumatoid factor affects the accuracy of immunoassays. By treating human blood-derived samples with a rheumatoid factor blocker before electrochemical detection of TB biomarker antibodies, the specificity of the assay is improved as the rheumatoid factor blocker inhibits rheumatoid factor present in the sample. The use of a rheumatoid factor blocker removes the need for an antibody binding inhibition step with MA-coated antibody capturing particles prior to electrochemical end-point detection of antibodies, as was previously required. This simplifies the test and results in a direct binding assay that successfully distinguishes between TB-positive and TB-negative patient sera, which was not achieved in standard ELISA immunoassays.

The sample may be incubated with a rheumatoid factor blocker at room temperature for up to 15 minutes.

The chemical blocking of the mycolic acid-coated electrode surface may include incubating the electrodes in an aqueous solution of up to 1% casein hydrolysate for more than 16 hours at room temperature.

The redox buffer may be 0.05 to 10 mM hexacyanoferrate.

The redox buffer may contain the same protein hydrolysate, and preferably at the same concentration, as characterised in accordance with the seventh aspect of the invention.

The potentiostat may be any potentiostat with EIS capability, preferably a handheld potentiostat to facilitate point of care use.

The diluted rheumatoid factor-blocked sample may be between a 1 in 100 and 1:1600 dilution of serum in 0.05 to 10 mM of [Fe(CN)₆]⁴⁺/[Fe(CN)₆]³⁻ (hexacyanoferrate redox probe) in phosphate buffered saline.

The screen-printed electrode may be any carbon electrode used for impedimetric analysis.

The screen-printed electrode may be a disposable electrode.

The screen-printed electrode may be exposed to the mycolic acid solution for a period of time to allow for cooling the heated solution, but not for so long that micelles drop out of solution.

According to a tenth aspect of the invention is provided a diagnostic kit for diagnosing tuberculosis in a human or animal subject by electro-impedance spectroscopy, the kit comprising a screen-printed carbon electrode according to the fourth or eighth aspects of the invention.

More specifically, according to the tenth aspect of the invention, there is provided a point of care tuberculosis diagnostic kit, which comprises: an individually packaged, mycolic acid antigen-coated, screen-printed carbon electrode according to the fourth or eight aspects of the invention; a rheumatoid factor blocking reagent to be added to samples; optionally a dried protein hydrolysate for blocking of non-specific antibody binding sites of the mycolic acid coated electrodes; a redox buffer solution; and standard equipment for measuring electro-impedance spectroscopy.

The mycolic acid antigen-coated screen-printed carbon electrode may be an electrode as prepared according to the third aspect of the invention, i.e. in accordance with the fourth aspect of the invention.

The protein hydrolysate may be used to produce a chemical blocking solution which may be as characterised with reference to the seventh aspect of the invention, preferably comprising 0.1 to 3% w/v protein, e.g. casein, hydrolysate. If the substrate has already been subjected to non-specific antibody binding site blocking according to the seventh aspect of the invention, then the protein hydrolysate may be omitted. Otherwise, it would typically be included.

The redox buffer solution may be 0.05 to 10 mM hexacyanoferrate redox probe for optimization of electrical signal strength, while minimizing reagent use.

The standard equipment may be known to people skilled in the art of electrochemistry and may include a portable or desktop potentiostat equipped with software to accumulate electrochemical signals.

The invention also extends, as an eleventh aspect thereof, to use of a polar organic solvent in preparing a solution of mycolic acid antigens for immobilising mycolic acid antigens on a substrate for binding of anti-mycolic acid antibodies by the antigens.

The invention also extends, as a twelfth aspect thereof, to use of acetone in preparing a surface modifying monolayer-free screen-printed carbon electrode for immobilisation of antigen thereon in the absence of a surface modifying monolayer.

The invention also extends, as a thirteenth aspect thereof, to use of a protein hydrolysate in blocking non-specific binding sites on a substrate comprising antigens or antibodies immobilised thereon. Wherein the protein hydrolysate may be casein hydrolysate.

Such uses, of the eleventh, twelfth and thirteenth aspects of the invention may be in accordance with the corresponding method aspects of the invention.

Using a polar solvent, such as acetone, is advantageously less aggressive than n-hexane, which is conventionally used for mycolic acid dissolution, and, in accordance with the present invention, surprisingly obviates the need for a hydrophobic, priming ODT self-assembled monolayer.

Custom-manufactured gold electrodes were previously required because they are solvent resistant and provide a strong sulphur-gold bond with ODT to form the self-assembled monolayer. As this is not required through the development of the present invention, carbon electrodes can be efficiently used as a more economical and eco-friendly alternative to gold electrodes.

An additional benefit of carbon electrodes over gold is that they do not require any mechanical polishing, only a pre-treatment step to remove organic impurities which the present invention unexpectedly succeeded in achieving using acetone. These factors provide the required reproducibility of the electrodes for the assay.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 shows, for the Examples, the electrical impedance spectroscopy (EIS) readings on carbon electrodes from manufacturer unmodified, treated with sodium carbonate, and treated with acetone for 5 minutes, error bars represent the standard deviation, n=3, in accordance with an aspect of the present disclosure;

FIG. 2 shows, for the Examples, the thin layer chromatography profiles of the upper and lower phase fractions in selected tubes of the countercurrent distribution tube train during the purification of mycolic acids from a crude extract of M. tuberculosis, A is a compound with Rf 1, B are compounds with Ratio to Front (Rf) of 0.6, C are compounds with Rf 0.1, lane=tube number/CCD upper (U) or lower (L) phase solvent, in accordance with an aspect of the present disclosure;

FIG. 3 shows, for the Examples, how the solubility of MA in acetone was determined by immunoassay (indirect ELISA) using monoclonal antibody (gallibody) to evaluate the amount of MA remaining in acetone solution for 1 hour and 4 hours after cooling from 90° C. to room temperature, compared to pure MA of the same initial concentration, error bars=standard deviation, n=3 (biological repeats), in accordance with an aspect of the present disclosure;

FIG. 4 shows, for the Examples, the feasibility of spotting of MA in acetone (M/A) compared to MA in hexane (M/H) on three substrate types: nitrocellulose, filter paper and TLC silica plates, determined by immunoblot test with monoclonal antibody against MA. Spots for MA in hexane are indicated by arrows labelled with ‘*’, spots for MA in acetone indicated by arrows labelled with ‘X’, darker spots on (A) nitrocellulose indicate indentations, in accordance with an aspect of the present disclosure;

FIG. 5 shows, for the Examples, a comparison of antigenicity of MA in acetone (M/A) with MA in hexane (M/H) spotted on silica TLC plate and developed with an anti-MA monoclonal antibody in an immunoblot test, spots for MA in hexane are indicated by arrows labelled with ‘*’, spots for MA in acetone indicated by arrows labelled with ‘X’, in accordance with an aspect of the present disclosure;

FIG. 6 shows, for the Examples, the change in light absorbance at 420 nm of MA in acetone at 0.25 mg/ml during cooling from 85° C. to indicate the dynamic micellar nature of MA in acetone solution, in accordance with an aspect of the present disclosure;

FIG. 7 shows, for the Examples, the change in light absorbance at 420 nm of MA in acetone at 0.125 mg/ml during cooling from 85° C. to indicate the dynamic micellar nature of MA in acetone solution, in accordance with an aspect of the present disclosure;

FIG. 8 shows, for the Examples, the change in light absorbance at 420 nm of MA in acetone at 0.05 mg/ml during cooling from 85° C. to indicate the dynamic micellar nature of MA in acetone solution, in accordance with an aspect of the present disclosure;

FIG. 9 shows, for the Examples, dynamic light scattering indicating the change in micelle size (nm) versus time (min) of cooling at 25° C. after heating at 85° C. of a solution of MA at 0.1 mg/ml in acetone, the first data point at t=0 is a blank acetone sample, the second datapoint at t=0 is the MA in acetone measured immediately after removal from the 85° C. heat source, in accordance with an aspect of the present disclosure;

FIG. 10 shows, for the Examples, the size (in nm) of micelles formed within 3 min from removal from heat at 85° C. of a 0.1 mg/ml MA in acetone solution to end-point temperatures over a range decreasing from 37-20° C., measured by dynamic light scattering, error bars represent the standard deviation, n=10, in accordance with an aspect of the present disclosure;

FIG. 11 shows, for the Examples, electrodes pre-treated with acetone, coated with 0.1 mg/ml MA in acetone solution and non-specific antibody binding sites blocked with 1% casein hydrolysate (CasH), error bars represent the standard deviation, n=12 before and after acetone pre-treatment (Ac wash), n=11 for MA with casein hydrolysate, in accordance with an aspect of the present disclosure;

FIG. 12 shows, for the Examples, a comparison between a TB-positive and a TB-negative serum at 1 in 1000 and 1 in 200 dilutions, MVT112/116 at 1 in 1000 (1,3-fold diff) and 1 in 200 (1,7-fold diff), MVT034/MVT225 at 1 in 1000 (1,9-fold diff) and 1 in 200 (2,2-fold diff), n=6 for TB positive serum and n=5 for TB negative serum, in accordance with an aspect of the present disclosure;

FIG. 13 shows, for the Examples, the benefits between the previous electrode coating process and the improved system using screen-printed carbon, in accordance with an aspect of the present disclosure;

FIG. 14 shows, for the Examples, the advantage of using casein hydrolysate as chemical blocking agent of MA coated electrodes (used as baseline) compared to PVP, impedance values for TB− and TB+ patient sera on carbon electrodes coated with 0.1 mg/ml MA in acetone solution and blocked with either 0.01% PVP or 0.01% casein hydrolysate are shown, corrected for baseline, error bars represent the standard deviation, n=4 for both MA with 0.01% PVP and 0.01% casein hydrolysate, error bars represent the range between duplicate values for TB-positive serum on both PVP and casein hydrolysate blocked electrodes and for TB-negative serum on both PVP and casein hydrolysate blocked electrodes, in accordance with an aspect of the present disclosure; and

FIG. 15 shows, for the Examples, the advantage of mixing a solution of rheumatoid factor blocker into serum samples to improve the resolution between TB+ and TB− patient sera with EIS analysis, the comparison of impedance values for TB− and TB+ serum with and without rheumatoid factor blocker on carbon electrodes coated with 0.1 mg/ml MA in acetone solution and blocked with 1% casein hydrolysate is shown, for all impedance measurements n=2, in accordance with an aspect of the present disclosure.

### DETAILED DESCRIPTION FOR CARRYING OUT THE INVENTION

Generally stated, disclosed herein is a method of detection of TB biomarker antibodies is illustrated by way of selected examples. These examples demonstrate how limitations of previously published inventions have been overcome, specifically regarding the manufacture of functional MA-coated electrodes for use in an assay in which MA antigens are presented as antibody biomarker capture agents.

In the examples provided herein, and in the specification generally, the following abbreviations are used from time to time:
MA=mycolic acid
EIS=electrical impedance spectroscopy
TB=Tuberculosis
Mtb=Mycobacterium tuberculosis
POC=point of care
ELISA=enzyme-linked immunosorbent assay
LFIA=lateral flow immunoassays
IgG=Immunoglobulin G
IgM=Immunoglobulin M
AmB=amphotericin B
RF=rheumatoid factor
RFB=rheumatoid factor blocker
Fc=fragment crystallizable regions
MARTI=Mycolic acid Antibody Real Time Inhibition
SPR=surface plasmon resonance
SPEs=screen-printed electrodes
ODT=octadecanethiol
PLGA=poly(lactic-co-glycolic acid)
Rf=Ratio to Front
U=upper
L=lower
M/A=MA in acetone
M/H=MA in hexane
CasH=casein hydrolysate
Ac wash=acetone pre-treatment
PBS=phosphate buffered saline
OD=optical density
CCD=countercurrent distribution
DLS=dynamic light scattering
PVP=polyvinylpyrrolidone

### Example 1

### Pre-Treatment of Carbon Electrodes

To minimise interelectrode variation and improve reproducibility of EIS signals, three groups of carbon electrodes were tested: unmodified electrodes, electrodes treated with Na₂CO₃, and electrodes pre-treated with acetone. Electrodes were exposed either to aqueous saturated Na₂CO₃ and electrochemically cycled between −0.4 and 0.4 volts three times at 23° C., or electrodes were submerged in acetone for between 2 and 7 minutes, in this case 5 minutes, at 23° C. and then rinsed with 500 μl of acetone. Impedance data was generated using carbon electrodes exposed to redox buffer hexacyanoferrate (0.05 mM potassium hexacyanoferrate (II) and potassium ferricyanide [Fe(CN)₆]⁴⁻/[Fe(CN)₆]³⁻) in phosphate buffered saline (PBS) containing 3.8 mM sodium azide and 1 mM EDTA, as described in Example 4, without addition of the stabilising agent.

### Results

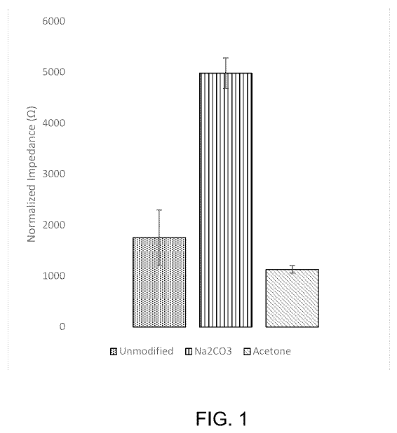

The data (FIG. 1) show that the treatment with acetone successfully reduces the interelectrode variation as indicated by the reduction in size of the error bars when electrodes are pre-treated with acetone. As expected, effectiveness over time is a distribution but the exposure time of acetone was titrated to an optimum level of about 5 minutes, although the effect may be achieved in the range of 2 minutes to 7 minutes.

### Conclusion

Pre-treatment of screen-printed carbon electrodes with acetone improves the interelectrode reproducibility in preparation for MA coating.

### Example 2

### Solubility and Antigenicity of Mycolic Acids in Acetone

### 1. Materials and Methods

### 1.1 MA Purification

### 1.1.1. Saponification of Crude Bacterial Extract

To release MA from mycobacterial cell walls, homogeneous bacterial cell pellets cultured according to conditions established by Ndlandla et al. (6) were re-suspended in 300 ml of saline. Re-suspension of the bacteria was allowed to sediment to the bottom of a Schott bottle before 250 ml of the saline was removed. The bacterial cells were re-suspended in the remaining saline and divided into equal parts before centrifuging for 20 min at 1500× g. The remaining saline was removed by decanting, making sure not to disturb the bacterial pellet that remains. The pellets were re-suspended in reagent A to obtain an optical density (OD) approximating McFarland standard 4, i.e., an OD of 0.837 at 486 nm measured using a spectrophotometer.

Saponification was done using autoclavable 2.5 L tightly-capped Schott bottles fitted with red thermally stable caps and rings. The Schott bottles were wrapped in foil and incubated at 70° C. overnight in a water bath covered in foil.

### 1.1.2. Crude Extract of Mycolic Acids

A funnel extraction was done on the saponified cell suspension in reagent A to obtain the crude MA extract. After cooling to room temperature, 1.5 ml of reagent B (HCl, (32% (v/v)), dissolved in double-distilled deionised water (dddH₂O) in a 1:1 volume ratio) was added for each 2 ml used of reagent A (KOH (25% (m/v) dissolved in methanol-water in a 1:1 volume ratio). The cell suspension was shaken carefully in a 2.5 L reagent bottle until no fumes were seen, then vigorously to ensure uniform mixing. The pH of the suspension was adjusted to 1 using reagent B to protonate all MAs for dissolution in chloroform. Chloroform (600 ml) was added before the suspension was vigorously shaken for 10 min in tightly capped reagent bottles. The crude MA extract was transferred to a separating funnel and allowed to separate into two phases at room temperature overnight. The lower phase containing the crude MA extracts was collected and evaporated at 60° C. in a pre-weighed round bottom flask using a rotary evaporator. Acetone (5-10 ml) was added to allow complete drying of crude MA extract. This was repeated until the crude MA extract was completely dry. The dried crude Mas were stored at 4° C. until further use.

1.1.3. Crude Extraction of MA in the Phase Solvent without Saline

Chloroform, methanol and water were mixed in a ratio of 42:39:19 in a 2.5 L reagent bottle. The solvents were vigorously shaken and allowed to form 2 phases in a separating funnel. Both phases were collected and stored separately. The crude MAs extracts were reconstituted in equal volumes of lower phase solvent and upper phase solvent. The phases were mixed together and transferred to a separating funnel, shaken vigorously and allowed to separate completely at room temperature. The lower phase extract was collected and evaporated at 60° C. in a pre-weighed round bottom flask on the rotary evaporator. Acetone (5-10 ml) was added to allow complete drying of crude MA extract. This was repeated until crude MA extract was completely dried. The crude MAs were stored in a desiccator, with indicator silica gel as the drying agent, overnight at room temperature to ensure removal of all water before weighing. Crude MA extract was stored at 4° C. until further use.

### 1.1.4. Purification of MAs by Countercurrent Distribution

The phase solvent used for countercurrent distribution (CCD) was prepared by mixing chloroform, methanol and 0.2 M sodium chloride in a ratio of 42:39:12. The solvent was allowed to form two phases in a separating funnel at room temperature. Each phase solution was retrieved and stored separately.

The crude MA extract was purified using the countercurrent distribution (CCD) method previously described by Goodrum et al. (14). The CCD instrument is built using an array of positions representing a chain of test tubes and comprises four racks of interlinking glass tubes.

The principle is as follows: each tube is filled with equal proportions of each phase (upper phase solution and lower phase solution). The lower phase of the two-phase solvent system is the “stationary phase”, whereas the upper phase is the “mobile phase”. The process begins at tube 0 which contains the mixture of substances to be separated in the lower phase solvent and all the other tubes contain equal volumes of the same solvent. The upper phase solvent is added to tube 0, where equilibration takes place, and the phases are allowed to separate. The upper phase of tube 0 is then transferred to tube 1 and fresh solvent is added to tube 0, after which the phases are equilibrated again. After each separation cycle, the clear upper phase moves along the tube rack from the first tube to the adjacent tube. Clean upper phase solution is added to the first tube at each transfer cycle, meaning that the initial volume of upper phase added to tube 0 will end up in tube 60 after 60 cycles.

Before purifying the crude MAs, the tubes of the CCD were cleaned by running the apparatus in wash mode sequentially with 1 L of 10% Contrad™ solution, 2 L of dddH₂O to rinse out the Contrad solution and 1 L of 96% ethanol to rinse the tubes. The machine was left upside down to drain all the liquid and to ensure it was completely dry before use. The apparatus was loaded first with lower phase in each tube. The crude MA extract was loaded into the first tubes of the CCD apparatus. The crude MA sample flask was rinsed with lower phase with saline to ensure quantitative transfer of MAs into the CCD apparatus. The crude MA sample solution fills the first 2 tubes of the CCD and volumes were adjusted to exactly 10 ml in both tubes using clean CCD lower phase solvent. Lower phase was added to first tube to equilibrate to the third tube and then re-adjusted to 10 ml and equilibration to fourth tube. In this way, the volume per tube was 10 ml for the first 4 tubes and approximately 5 ml was left over in the fifth tube after equilibration.

CCD upper phase solvent (650 ml), enough for 60 cycles, was loaded to the large upper phase tank of the machine. A volume of 10 ml of upper phase CCD solvent with saline was transferred manually to tube 0. Every second tube after the fifth tube was then loaded with 10 ml CCD lower phase solvent. The contents were mixed manually 20 times by shaking the machine and the contents were allowed to separate into two phases at rest position. This was the first cycle. Clean upper phase was transferred to the first tube to commence the second cycle and the routine was repeated until 60 cycles were done. In the first tubes initially, the upper phase absorbed some of the lower phase and the absorption was corrected by adding clean lower phase CCD solvent with saline to tube 0 at every cycle until it was no longer necessary. The volume of upper phase was adjusted to exactly 10 ml by adjusting the rod that controls the volume of upper phase entering the tubes.

### 1.1.5 Analysis of CCD Purified MAs Using TLC

To analyse the purity and distribution of the MAs, 10 ml of lower phase and 10 ml of upper phase samples of the same tube for every second tube of the 60 tubes were spotted on TLC plates.

TLC is carried out as follows. MA samples (10 ml) were loaded on aluminum-backed TLC silica plates by spotting using a Hamilton syringe on a pencil line drawn 1 cm from the bottom of the silica plate. The spots were left to dry at room temperature. Once the spots were dry, the silica plate was placed in the TLC developing tank with running solution of diethyl ether (30% (v/v)) dissolved in hexane which was drawn up the plate via capillary action until it reached to about 2-3 cm from the top of the TLC plate. Subsequently, the plate was immersed in the visualisation reagent container for 5 seconds, in this case a developing solution. The plate was removed from the developing solution using tweezers, placed on laboratory tissue paper and charred immediately using a heat gun. The plates were analysed, and retention factor values were calculated. From this, the MA was identified in tubes 0-15. Both lower and upper phases of tubes 0-15 were withdrawn from the CCD tubes, pooled and evaporated at 80° C. until mostly dry.

### 1.1.6. Acetone Precipitation of CCD Purified MAS

The salt from the saline must be removed in order to obtain correct yields of pure MAs. The semi-dried MA was mixed in 2 ml chloroform and 1 ml water and shaken vigorously before transferring to a separation funnel allowing the mixture to separate into two phases at room temperature. The upper phase containing saline was discarded and the lower phase evaporated at 80° C. using a rotary evaporator. The saline removal process was repeated 3 times. Acetone (5-10 ml) was added to allow complete drying. This was repeated until MA was completely dried.

To remove contaminants that are attached or associated with CCD purified MAs, the dried MAs from the previous step were dissolved in 4 ml chloroform and concentrated to 1 ml at 85° C. on the rotary evaporator. This was done for 5 minutes to ensure all MAs were dissolved. The addition of acetone was done to precipitate the MAs out of solution. To the MA extract, 20 ml of acetone was added and heated at 85° C. in a pre-weighed round bottom flask. The MAs were allowed to precipitate from the acetone solution overnight at 4° C.

Filtration through filter paper previously rinsed with chloroform and acetone was used to separate the MA precipitate from contaminants. Cold acetone (10 ml) was used to rinse the MA precipitate on the filter paper. The precipitate was trapped onto the filter paper and the acetone filtrate filtered through into a round bottom flask labelled “acetone filtrate/MA supernatant”. This was repeated three times until the flask that contained the MA precipitate was completely clean. The precipitate on the filter paper was then rinsed in 10 ml chloroform three times into a pre-weighed round bottom flask labelled “MA precipitate” before it was evaporated at 60° C. using a rotary evaporator until completely dried. The MA precipitation from contaminants was repeated a second time. Acetone (100 ml) and chloroform (4 ml) were added to the MA precipitate recovered from the filter paper and heated at 85° C. for 5 minutes. The MAs were allowed to precipitate overnight from the acetone solution at 4° C. and recovered as before.

The MA precipitate and supernatant that were collected in round bottom flasks were stored in a desiccator, with indicator silica gel as the drying agent, overnight to ensure removal of all water before weighing the flasks using an analytical balance.

It is important to note that the use of acetone described in sections 1.1.2 to 1.1.5 is not the use of acetone as characterised according to the invention, but a more conventional use of acetone in the art for precipitation.

### 1.2 Solubility of MA in Acetone

To determine the extent to which MA can be solubilised in acetone, acetone was added to an excess amount of MA, solubilised by heating and cooled down to room temperature to provide a saturated solution of MA in a vial at room temperature that also contains insoluble MA adhering to the walls that formed during cooling. The soluble and insoluble MA were quantified by immunoassay using recombinant monoclonal antibodies developed by Ranchod et al. (2).

### 1.2.1 Purification of Gallibodies

Gallibody clone type 12CH1-4 was purified according to Ranchod et al. 2018 (2) using nickel affinity columns. 5 ml of cell culture media containing the gallibodies was diluted with 45 ml 1× lysis buffer before being passed through the column. The eluent was passed through the column a second time, before discarding the eventual flow-through. The column was washed twice with 1× lysis buffer and proteins retained in the column were eluted in 1 ml fractions using the elution buffers 1, 2, 3 and 4, in that order. Borate buffer was added in a 1:1 ratio to the purified gallibodies before concentrating by ultrafiltration-centrifugation at 3500×g for 20 minutes using Vivaspin 10 000 MW PES centrifugation units (VivaScience, Satorius Group, United Kingdom). Protein concentration was determined by Bradford assay.

### 1.2.2 Determination of the Solubility of MA in Acetone

To characterise the solubility of MA in acetone, 2 vials (i and ii) each containing 1 mg MA and 2 ml of acetone were heated for 5 minutes at 90° C. on a Reacti-Therm III heating block. Each vial was left at room temperature to cool after mixing. After 1 hour, the MA/acetone solution of the first vial (i) was transferred by pipetting to a clean amber glass vial (iii). After 4 hours the MA/acetone solution of the second glass vial (ii) was transferred to a clean amber glass vial (iv). The four vials were placed on the heating block at 80° C. under flow of nitrogen gas to allow the contents of each vial to evaporate and dry. All vials were stored in a desiccator overnight at room temperature to allow complete desiccation before weighing.

Nunc-Maxisorp ELISA plates were coated with 50 μl purified MAs and the residual MAs from vials i-iv from the previous step, at a concentration calculated as if the original 1 mg of MA was still present in each vial. The residual MA in the vials was first dissolved in freshly distilled hexane to a decreasing dilution range 0.25-0.1 mg/ml. All samples of MA were coated in triplicate. Non-specific binding sites of each well were blocked with 300 ml per well of 4% (m/v) casein hydrolysate/PBS pH 7.4 for 2 hours at room temperature and then washed three times with 300 μl per well of PBS/0.1% Tween 20. The wells were then incubated for 1 hour at room temperature with 50 μl per well of gallibody 12CH1-4 diluted in 4% (m/v) casein hydrolysate/1×PBS-0.1% Tween 20 pH 7.4 to a concentration of 0.031 mg/ml. Unbound antibodies were removed by washing the wells with PBS/0.1% Tween 20 as above. Secondary antibody conjugate (Goat anti-chicken Fc: HRP) (AbD Serotic, Kidlington, UK) was diluted 1:1000 in 4% casein hydrolysate/1×PBS-0.1% Tween 20, pH 7.4 before adding 50 ml to each well and the plates incubated for 1 hour at room temperature. The conjugate solution was discarded, and the plates washed as above. The signal was developed by adding 50 μl of TMB Single Solution Chromogen for ELISA to each well and then incubated at room temperature for 5 min. To stop the reaction, 50 μl of 1 M H₂SO₄ was added to each well. Plates were read at 450 nm.

### 1.3 Antigenicity of MA in Different Solvents: Acetone Compared to Hexane

### Immuno-Blot Test of Acetone Soluble MAs

A volume of 4 ml acetone was added to an aliquot of 1 mg MA and heated at 90° C. on a heating block for 5 min and shaken by hand to mix. The MA/acetone solution was left to cool for 1 hour before 10 μl is spotted using a Hamilton Syringe on a silica plate at a concentration of 0.25 mg/ml. Hexane (4 ml) was used to dissolve another aliquot of 1 mg/ml MA that was also spotted as above and left to dry for 30 min. The silica plate with spotted MA was placed in a petri dish and immersed in 4% (m/v) casein hydrolysate/PBS pH 7.4 such that the entire plate is covered in solution. It was then incubated for 2 hours at room temperature. After 2 hours, the buffer solution was tipped out of the petri dish, making sure not to tip out the silica plate, which was then washed three times with PBS/0.1% Tween 20 by gently tilting the buffer solution side to side in the petri dish without removing the silica plate. The buffer solution was tipped out and excess buffer was removed with a disposable plastic dropper. The silica plate was then immersed in gallibody 12CH1-4 solution diluted in 4% (m/v) casein hydrolysate/1×PBS-0.1% Tween 20 pH 7.4 to a concentration of 0.031 mg/ml and incubated for 1 hour at room temperature. After washing as before, secondary antibody solution (Goat anti-chicken Fc: HRP) (AbD Serotic, Kidlington, UK) diluted 1:1000 in 4% casein hydrolysate/1×PBS-0.1% Tween 20, pH 7.4 was added to the plate in the dish, followed by incubation for 1 hour at room temperature. The plate was washed three times with PBS/0.1% Tween 20 to remove unbound secondary antibody before 5 ml of TMB blot solution was added to develop the colour for 3 minutes, after which the colour signals on the plate were analyzed visually and recorded by taking a photograph using a mobile phone camera. Because TMB oxidation is fast, colour development must be recorded within 5 minutes, after which precipitation of the coloured stain occurs randomly, reducing the resolution.

### 2. Results

### 2.1 Countercurrent Purification of Mycolic Acids

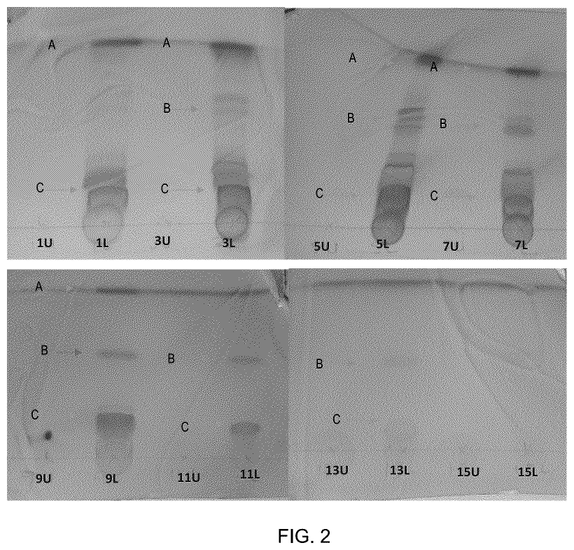

To analyse the distribution of MAs after CCD purification, TLC was performed from tubes 0-60 using both upper and lower phase, from every second tube starting with tube 1 to tube 15. The TLC results are shown in FIG. 2.

It can be seen from FIG. 2 that MA spots at Rf 0.1 were visible from tubes 1 to 13 and peaked in tube 5L. The compound with Rf of 1 also peaked at tube 5L and started to fade away as the MA spot started fading from tube 7L, showing an association with the MAs and the lower phase solvent. The compound with Rf of 0.6 peaked in tube 5L and faded after tube 13L. As a similar spot was seen at the same Rf of 0.6 in the lane that only contained the lower phase solvent, it is speculated to originate from the lower phase solvent. The dark spot at the origin that peaked in tube 5L is speculated to be an ionised (more polar) form of MA. Nothing was seen in the upper phase of each tube, which therefore makes chloroform the most probable culprit for contributing the Rf 0.6 contaminant in the fractions that were harvested from CCD. The absence of material in the upper phase showed that most contaminants from the crude MA extract moved on. No spots for MA or contaminants were seen from tubes 15-60.

MAs display low mobility on TLC, as they do not move far from the origin. Accordingly, FIG. 2 shows that MAs also did not move along in the counter current tube train, but were retained in the first tubes, showing that they are extremely hydrophobic. More hydrophilic substances move further along the train of tubes. Even though FIG. 2 shows only three compounds/contaminants excluding MAs, most of the contaminants of the crude MA extract seen in the TLC chromatogram of FIG. 2 would have moved on down the rest of the tubes in the tube train. CCD purification at a lower scale had crude extract loaded in the first tube only and yielded MA enriched sample in tubes 1-10. Upon the up-scaling of the sample load of crude extract, the MA containing fraction was collected from tubes 1-15, due to the loading of the sample over 5 tubes, instead of one. The efficiency of separation therefore was not affected by up scaling CCD purification, while gaining the benefit of improved MA detectability in the CCD tubes by TLC to assist in identification of the MA in the tube train after separation.

### 2.2 Determining the Limit of the Solubility of MA in Acetone

To determine the acetone solubility of MA in acetone, the gallibodies were applied in quantitative ELISA-immunoassay in order to avoid the inaccuracy inherent to the gravimetric determination of the small amounts of MA that can be transferred from a vial in which MA was solubilised in acetone under high temperature and then allowed to cool for a determined time before transfer to a fresh vial.

It is reasonable to envision the physiochemical properties of the functional groups of MA in solution having conformational freedom of folding the long methylene units in the backbone of MA under high temperatures as it is known that in spite of their high molecular weight, mycolic acids have relatively low melting points and are highly soluble in non-polar organic solvents such as chloroform, hexane, heptane, methylene chloride, toluene and xylene (15). It therefore stands to reason that the MA can be solubilised under high temperatures in almost any non-polar solvent.

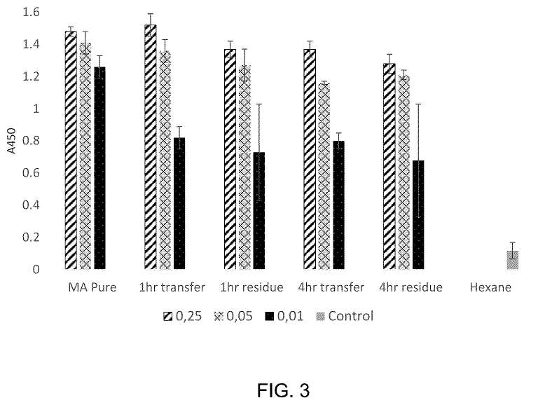

Two vials of MA (1 mg each) were set up, the MA dissolved in acetone (2 ml), heated at 90° C. for 5 minutes and shaken by hand before leaving to cool for either 1 hour (vial 1) or 4 hours (vial 2). After cooling, the contents of vials were transferred into separate clean vials. All vials were placed into a heating block at 80° C. in the flow of nitrogen gas to allow the contents to be evaporated to dryness. The dry vial content was subsequently dissolved in hexane to a dilution range of 0.25, 0.05 and 0.01 mg/ml to be analysed by ELISA using gallibody 12-1. Results are shown in FIG. 3.

From heating MA solution in acetone at 90° C. it is observed that MA remains in solution at 1 hour due to the clear solution that was observed. The solution became progressively opaque towards 4 hours after cooling, but the amount of MA that could be transferred remained at around 50% of the original content. The increasing opaqueness with time is most likely due to micellar MA dynamically growing bigger and eventual sedimentation on the bottom of the vial. This means that a solution of MA in acetone is not stable at saturating concentrations and should be applied within 4 hours after cooling or used at a lower concentration.

From the results in FIG. 3 it can be seen that the amount of MA that could be transferred and the amount that was left behind (residue) were approximately the same for both 1 hour and 4 hours of cooling. This is also true for antigenicity as the ELISA signal is comparable and almost the same as pure MA at the same concentration.

From this it was concluded, surprisingly, that MA can be dissolved in acetone at room temperature to a certain limit, of which the first indication is an upper limit of about 0.25 mg/ml. However, MA micellar solubility in acetone over a period of at least four hours after cooling is unstable.

### 2.3 Immuno-Blot Test of Acetone Soluble MAs

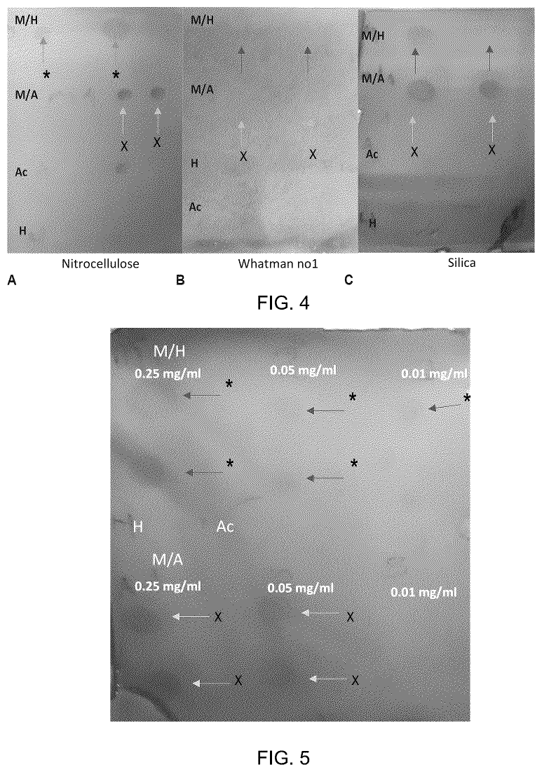

A 10 μl aliquot of MA in acetone at 0.25 mg/ml was blotted in duplicate on nitrocellulose to demonstrate the effect of incompatibility with acetone. Similarly, MA in hexane at the same concentration was blotted in duplicate on the same substrate. It was compared to two different substrate types, i.e., Whatman no 1 paper and a silica TLC plate that were blotted with MA in the same way as the nitrocellulose substrate. Hexane and acetone alone were spotted as negative controls. The blotted and dried substrates were incubated in gallibody type 12-CH1-4 at a concentration of 0.031 mg/ml. Immunoblot development with secondary antibody-HRP conjugate gave the results shown in FIG. 4. Blue spots indicate positive binding of gallibody to the MA.

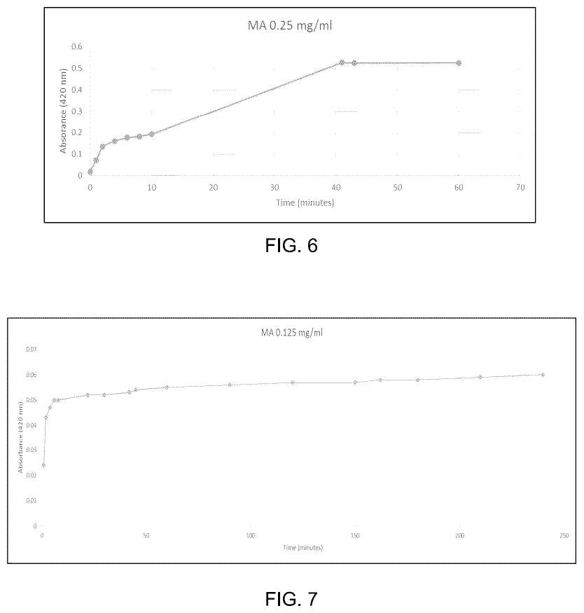

The results of the immunoblot test (FIG. 4) show that where MA in acetone was spotted on nitrocellulose, the nitrocellulose was dissolved at the area of spotting, confirming incompatibility, in contrast to where MA was spotted from hexane solution (FIG. 4A). The Whatman no 1 filter paper substrate, was not usable as no spots were visible (FIG. 4B). Results of blot test on the silica TLC plate show spots for MA in acetone and MA in hexane (FIG. 4C), however only one spot of MA in hexane was visible, whereas both spots were visible for MA in acetone. The positively visible signal is far stronger for MA in acetone than MA in hexane. The negative controls (hexane and acetone only) showed no detectable signals. The MA signals in FIG. 4C indicate that MA is antigenic when spotted onto silica and that MA in acetone remains antigenic, even to a greater degree than when spotted from hexane solution. To further investigate the antigenicity of MA in acetone compared to MA in hexane on silica, a dilution range was done at spotting concentrations of 0.25, 0.05 and 0.01 mg/ml. Results of the blot test of MA in acetone and MA in hexane spotted at decreasing concentrations are shown in FIG. 5.

MA concentration dependent positive antibody binding spots were visible for MA spotted in acetone and hexane at concentrations of 0.25 mg/ml and 0.05 mg/ml in FIG. 5, but significantly weaker for spots from MA in hexane, indicated by arrows labelled with ‘*’. The duplicate spots for MA in acetone arrows labelled with ‘X’ showed clear duplicate spots, which decreased in intensity with lower concentration, were more visible at concentration of 0.05 mg/ml compared to those coated with MA in hexane. The reproducibility of spotting MA in acetone on silica was also better than MA in hexane. The negative controls (hexane and acetone only as spotting reagents) showed no detectable signal, testifying to the success of washing away the background in the immunoblotting process.

These results confirm that aluminum backed silica plates work well as substrate for an immunochemical detection of MA spotted on the silica. Clear antibody binding spots were obtained where MA was spotted from acetone solution, while the background of unspotted silica could be washed clear with aqueous buffer, indicating no non-specific binding of antibodies to silica. In addition, the silica plate was not damaged after extensive washing with aqueous buffer.

### Discussion

The use of MA to detect TB patient anti-mycolic acid antibody biomarkers towards a handheld device for diagnosis in a POC environment, which would fulfil a great need in the management and control of the TB epidemic, was investigated.

The availability of recombinant, monoclonal anti-MA gallibodies provided the opportunity to both quantify minor amounts of MA and characterise it in terms of antigenic properties. Previous work in this research area showed that the application of the gallibodies in immunoassay faced several challenges, mainly due to the nature of the large lipid MA antigens. MA could successfully be immobilised from hexane on nitrocellulose in an antigenic conformation (15). Even though this was a success, hexane is not compatible with the bio-printing machinery used for electrode coating. The dissolution of MAs in polar solvents such as acetone, surprisingly shown to be possible in accordance with the present invention, would solve this problem. Lipid immobilisation on nitrocellulose was not suitable due to the acetone incompatibility (16), while the typical low avidity of serum anti-lipid antibodies presented an almost unsurmountable problem of labelling (15).

MA was dissolved in acetone at high temperature and then allowed to cool. The amount of MA remaining in solution was investigated. FIG. 3 shows a quantitative result where MA was titrated down to where a decreasing signal was obtained, thus indicating comparable MA amounts in the transfer vials and what residue remained as insolubilities in the original vial.

It is assumed that the MA dissolves as dynamic micelles in acetone at high temperatures. It remained in solution for approximately 4 hours after cooling. Under these conditions MA solubility was found to be approximately 0.25 mg/ml and lower, probably as a micelle suspension that coagulates into a sediment after several hours at saturating concentration.

The important contribution of the present invention of solubility of MAs in a polar solvent, such as acetone, finds application in overcoming challenges in development of TB immunodiagnostics because of the chemical compatibility of acetone for antigen printing instruments, on electrodes and other substrates for MA immobilisation (16). However, acetone is incompatible with printing on nitrocellulose, which was shown in FIG. 4A, as the structural integrity of the nitrocellulose failed due to dissolution at the area spotted. Instead, TLC silica plates were found to be a suitable substrate on which to spot MA antigen from acetone solution as seen in FIG. 4C, also confirming MA antigenicity after spotting and immunoblotting. In FIG. 5 the superiority of acetone over hexane as MA solvents is proven, with darker spots developed from MA-acetone compared to MA-hexane. It appears that MA spotted from acetone is presented as a better antigen on silica than can be achieved with MA-hexane solutions. A possible explanation for this is the micellar nature of acetone solutions of MA, observed by the homogeneous opaqueness of the solutions, which gradually increased within four hours after heating. Hexane solutions of MA remained stably clear. It is believed that the micellar packing of MA assists their folding into an antigenic conformation before and during adsorption onto silica. The importance of the folding properties of MA into structures of variable antigenicity was demonstrated by Beukes et al. 2010 (17).

### Example 3

### Micellar Nature of MA Dissolved in Acetone

The newly disclosed solubility of MA in acetone, in accordance with the present invention, is exemplified in Example 2 above. MA appears to remain soluble in acetone at room temperature for a limited time after removal from heating at 90° C. The data suggests that, compared to hexane, the more polar acetone has the potential to better orientate the folding of MA towards the antigenic state when used as the solvent for MA to immobilise the latter onto a solid substrate such as silica.

No information was available in literature on MA dissolution in acetone, which warranted a characterisation study of how MA behaves in acetone solution. Work was therefore performed to:
Determine the limit of ‘solubility’ of MA in acetone, and
Characterise the behaviour of MA in acetone at different temperatures.

### 1. Methods

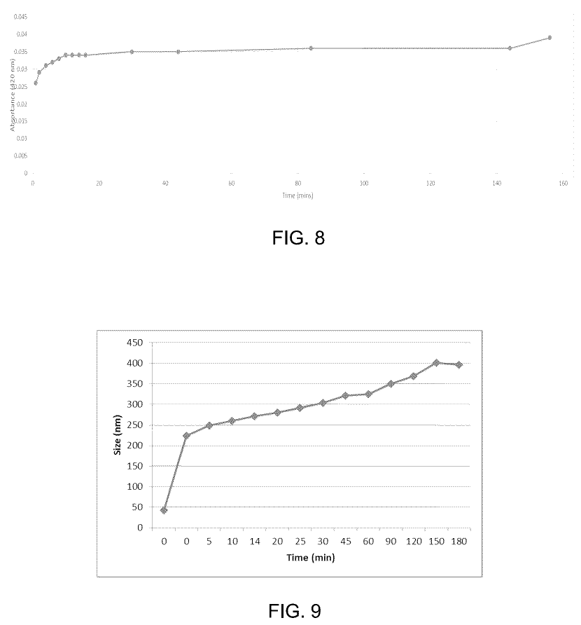

Determining the limit of ‘solubility’ of MA in acetone A spectrophotometric method was used to determine the limit of solubility of MA in freshly distilled acetone over the concentration range of 0.25, 0.125 and 0.05 mg/ml using a quartz cuvette (FIGS. 6, 7 and 8). Samples of MA were weighed and dissolved in acetone by heating to 85° C. for 5 min in a capped, amber glass vial.

Observation: The MA solution in the vial was clear immediately after the vial was removed from the heat source. Within one minute after the vial was removed from the heat source, the solution appeared turbid.

The MA solution was transferred from the glass vial to a quartz cuvette, the opening of the cuvette is covered with a PTFE cap, heated for one minute on a heat block at 85° C. and immediately transferred to a spectrophotometer where absorbance measurements are recorded at 420 nm, at intervals of 2, 15, 30 and/or 60 minutes at room temperature after removal from heat.

Observation: During transfer of the MA-acetone from the heat block in the quartz cuvette to the spectrophotometer, the solution appeared clear.

### 1.2 Characterise the Behaviour of MA in Acetone at Different Temperatures

The apparent change in absorbance over time at a concentration of 0.125 mg/ml suggest that the micelles may be in a dynamic state. Therefore, a concentration of 0.1 mg/ml MA in acetone was selected as the concentration at which to characterise the behaviour of MA in acetone for temperature-dependent studies.

MA in acetone solution having a concentration of 0.1 mg/ml was heated on a hot plate at 85° C. for 5 minutes. The vial was removed from the hot plate and allowed to cool on a laboratory bench for 5 minutes. Of the MA-acetone solution, 2 ml was transferred to a clean quartz cuvette with a rectangular, non-airtight, Teflon cap. This vial was heated for 1 minute on a hot plate at 85° C. and immediately placed in a Zetasizer receptacle. The Zetasizer equilibrates the temperature of the cuvette for 180 seconds using temperature-controlled airflow over the walls of the cuvette.

Dynamic light scattering (DLS) was used as the subsequent characterisation method due to its high resolution in detecting scattered and transmitted light. A Zetasizer Nano zs uses dynamic light scattering to provide data of the size (diameter) and the polydispersity index (size distribution) of nanoparticles.

To investigate the effect of change in temperature on dynamic light scattering of the MA in acetone solution, a solution of 0.1 mg/ml MA in acetone was heated to 85° C. in a sealed glass vial with a screw cap. The hot solution was then transferred into a clean quartz cuvette capped and heated at 85° C. for an additional minute and allowed to equilibrate inside the Zetasizer receptacle for three minutes across a range of decreasing temperatures. Each size measurement occurred at 50, 40, 37, 25 and 20° C.

### 2. Results

Neither acetone, nor solutions of MA, are significantly yellow of colour, therefore a solution of MA in acetone was not expected to exhibit a significant absorption of light at 420 nm wavelength. Should any absorption at 420 nm occur in acetone solutions of MA, then this can be assumed to be due to light scattering, indicating the particulate nature of the MA in solution, probably as micelles. In all the FIGS. 6-8 a rapid increase in light scattering was measurable during the first 10 minutes of cooling from 85° C., indicating the rapid formation of MA micelles in solution. Between 10 minutes and an hour, the rate of increase of absorption tended to level off towards a plateau, with the slopes of increasing absorption proportional to the MA concentration. Thus, the growth of the MA micelles during cooling from 85° C. is directly proportional to the initial concentration of MA in the acetone. At highest MA concentration of 0.25 mg/ml the solution was also the fastest to indicate visible turbidity, probably occurring from coalescing MA micelles that trend to precipitate out of solution after more than four hours of cooling.

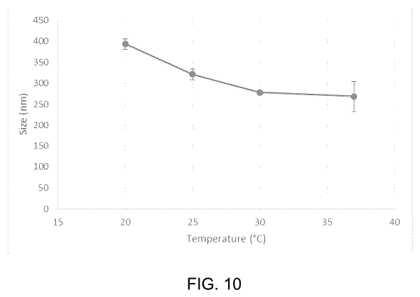

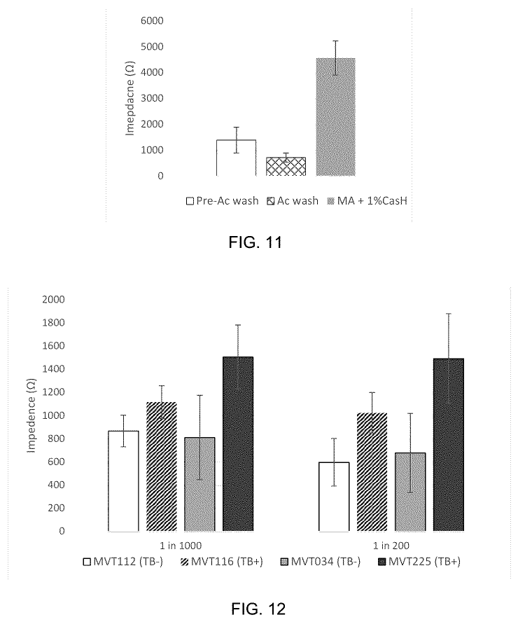

FIG. 6 shows that at 0.25 mg/ml the MA in acetone solution coalesces rapidly into growing micelles that the solution may be deemed too unstable for the purposes of reliable EIS electrode coating. FIG. 7 shows that at 0.125 mg/ml the MA in acetone solution indicate a relatively stable plateau of light scatter that increased only by 0.1 absorbance units between 10 minutes and four hours, indicating a relatively stable micellar solution that may suffice for the MA coating of electrodes. The data in FIG. 8 at 0.05 mg/ml MA concentration indicate the most stable plateau of light scatter after 10 minutes, but at a significantly lower plateau height, which may require greater volumes of solution for coating of electrodes.

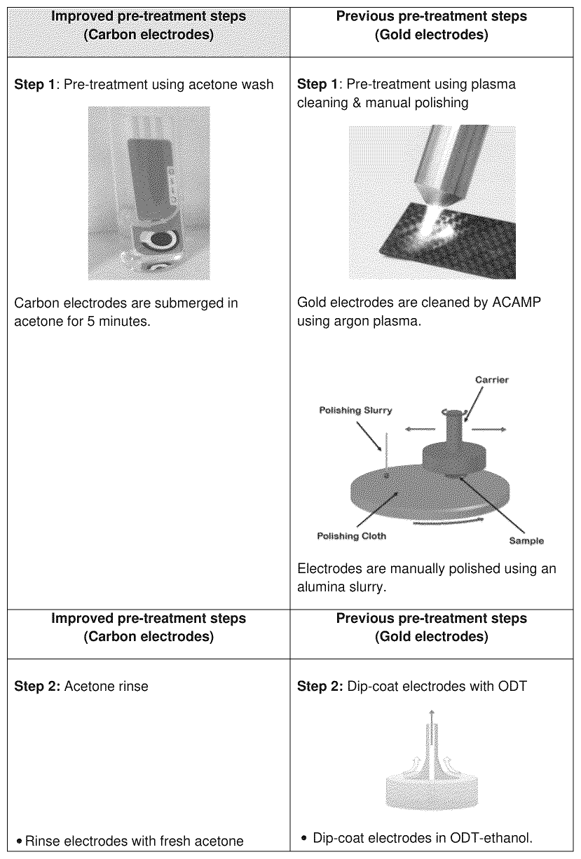

The data in FIGS. 6-8 indicate 0.1 mg/ml of MA in acetone as a practical concentration with which to coat electrodes with MA within four hours of cooling. For this reason, this concentration was selected to study the behaviour of MA in acetone at different temperatures. Dynamic light scattering allows the estimation of micellar size over time of a MA solution. In FIG. 9 significant logarithmic growth of micelles in a 0.1 mg/ml MA solution was observed in up to 3 hours of cooling from 85° C., reaching up to 400 nm in that time, but remaining in a visibly clear solution. The data indicated that the micelles increase in diameter over time from 225 nm (t=0 min) to 400 nm (t=180 min). As far as the inventors are aware, this is the first description of spontaneous micelle formation of mycolic acids in acetone.

A subsequent task was to investigate if temperature has an effect on the size of the micelles in MA solution. A solution of 0.1 mg/ml MA in acetone was heated to 85° C., transferred into a clean quartz cuvette, heated at 85° C. for an additional minute and allowed to equilibrate inside the instrument for 180 seconds across a range of decreasing temperatures.

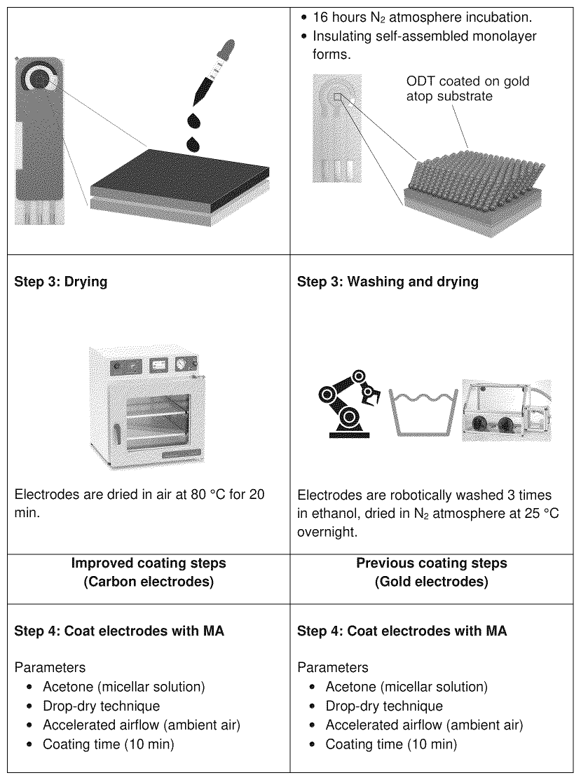

At 50° C. and 45° C., no dynamic light scattering was detected. At 37° C. the instrument detected signal indicative of micelles having an average size of 270 nm. When the temperature was decreased to 30° C., a decidedly narrow polydispersity index was obtained with nanoparticles having an average diameter of 280 nm. The size of the micelles appears to increase as the temperature decreases. The increase in micellar size can be observed in the data in FIG. 10 from ca. 270 nm at 37° C., to almost 400 nm at 20° C.

### 3. Discussion

The rapid increase in absorption/diffraction of light directly after removal of the MA-acetone solution from heat may be assumed to be due to micelle formation. The gradual increase in absorption/diffraction after 10 minutes is taken to be a gradual growth of micelle size, towards a state where the coalesced micelles tend to sediment out of solution after a few hours as the solution cools. By visual observation it is noted that sedimentation occurs faster at the highest concentrations of MA in acetone.

Previous work by Baumeister, Shaw and Verschoor (4) indicate that MA-presenting liposomes of a smaller size display better ability to present MA in an antigenic manner that is able to detect active TB biomarker antibodies.

### 4. Conclusion

There are several advantages to utilising acetone as a solvent for MA for the purpose of applying it to coat electrodes for detection of anti-MA antibodies, including improved antigenic orientation of the immobilised MA (Example 2). In addition, acetone is compatible with the antigen depositing or printing apparatus for the required upscaling of automated antigen coating of screen-printed electrodes. For coating of biosensor electrode surfaces, the behaviour of MA in micellar acetone solution points towards a MA concentration of around 0.1 mg/ml that is to be heated to 85° C. or higher to allow for complete chemical dissolution, which then needs to be allowed to cool to between 25° C. and 35° C. and applied to the electrodes within approximately 4 hours after heating to ensure the reliable coating of MA antigen in the desired conformation from a relatively stable MA micellar solution.

### Example 4

### Detection of TB Biomarker Anti-MA Antibodies in Human Serum Samples by Electro-Impedance Spectroscopy (EIS) on MA-Coated Carbon SPEs

Patients with active TB will exhibit increased levels of antibodies to mycobacterial cell wall mycolic acids. The antibodies have previously been detected by various techniques including ELISA, waveguide and resonant mirror biosensors. Results with ELISA published by Schleicher et al. (5) provided 57% accuracy. With a resonant mirror biosensor, 82% accuracy was achieved for detection of biomarker anti-mycolic acids antibodies (8). The latter approach took the form of an inhibition assay in the so-called MARTI assay described by Lemmer et al. (9). In order to meet the requirements of affordability and high through-put sample analysis, the MARTI-assay was adapted to allow the detection of anti-MA antibodies on screen-printed MA coated gold electrodes by means of an amperometric technique, namely EIS. The ability to distinguish between TB-positive and TB-negative sera occurs as a result of the measurement of a difference in bound antibody to a MA coated surface. Previous approaches were poorly reproducible in terms of the requirement of polishing of the gold electrodes before coating, the need for creating a self-assembled impedimetric lipid monolayer, the challenges with the use of the unstable dimethylformamide solvent for MA coating, and the instability of the PLGA nanoparticles used as immunosorbent in the pre-treatment of serum samples.

In this example, the above technical challenges are overcome by substituting screen-printed gold electrodes with commercially available screen-printed carbon electrodes, preparing these for MA coating by a simple short wash in acetone, and using MA dissolved in acetone at a concentration that it creates its own self-assembling impedimetric lipid structures that exhibit better antigenic properties than when coated from hexane solution. In addition, the method replaces the need for pre-treatment of serum samples with MA-coated nanoparticles by repressing any rheumatoid factor activity in the serum samples by addition of commercially available rheumatoid factor blocker solution.

### 1. Materials

Blood samples were provided by Department of Internal Medicine (Infectious Diseases), Faculty of Health Sciences, University of Pretoria. Patient consent as per the ethics clearance provided by the University Ethics Committee was obtained prior to collection of blood samples. Blood samples were allowed to clot for 4 hours, serum aspirated into clean 1.5 ml Eppendorf tubes, centrifuged at 4° C. to sediment red blood cells, serum (100 μl) aliquoted and stored at −80° C. until use.

Unless specified, all reagents are at least 99.5% pure. Distilled de-ionised water (dddH₂O) is used for the preparation of reagents and rinsing of screen-printed electrodes.

Materials: Acetone (min 99.5% purity), deionised water, disodium ethylenediaminetetraacetate dihydrate, disodium phosphate, absolute ethanol (99.8%), mycolic acid, potassium chloride, potassium phosphate monobasic, rheumatoid factor blocker (final concentration of 1 mg/ml), pre-dried silica gel, sodium azide, sodium chloride and sodium phosphate dibasic.

### 2. Methods

### 2.1 Preparation of Hexacyanoferrate Redox Buffer

Hexacyanoferrate (1 mM potassium hexacyanoferrate (II) and potassium ferricyanide [Fe(CN)₆]⁴⁻/[Fe(CN)₆]³⁻) were prepared in phosphate buffered saline (PBS) containing 3.8 mM sodium azide and 1 mM EDTA. The pH was adjusted if required to 7.44 with either 1 M HCl or 1 M NaOH. The solution was filtered using a Pall filtration system (Washington, USA) and 0.2 μm cellulose acetate filters. Casein hydrolysate is added as a stabilizing agent at 1% (m/v).

2.2 Pre-Treatment of Carbon SPEs with Acetone

Cyclic voltammetry (CV) and EIS data were generated using a potentiostat, device software and disposable well cells. Carbon electrodes were characterized using cyclic voltammetry and EIS. CV scans were cycled from −0.2 V to +0.4 V (E vertex1 to E vertex2), with a voltage step of 2.44 mV at a scan rate of 50 mV/s. EIS scan occurred at a dc voltage of 0.135 V, AC amplitude of 0.01 V at 50 frequencies from 2000 to 0.1 Hz. Electrodes were rinsed with dddH₂O and air-dried. The sensor surfaces were submerged in acetone for between 2 to 7 min and subsequently rinsed with approximately 500 μl of acetone. Electrodes were dried in an EcoTherm oven (Labotec, ZA) at 80° C. for 20 min and subsequently cooled in air for 5 min. CV and EIS data were then generated.

2.3 Coating of Carbon SPEs Using 0.1 mg/ml MA-Acetone

A glass micropipette (Blaubrand, Germany) mounted to the robotic arm of a surface plasmon resonance (SPR) biosensor (Metrohm Autolab, Netherlands) was used to dispense 3 μl of a 0.1 mg/ml MA in acetone solution at a temperature of between 25 to 37° C., in this case at 30° C., onto the working electrode of each SPE at 30° C. in 10 repeated deposition actions. Acetone was allowed to evaporate for two minutes, after which the electrodes are incubated under high vacuum≤50 mTorr using a Virtis lyophilizer (SP Scientific, USA) for at least 16 hours to remove trace acetone.

2.4 Blocking of Carbon SPEs with 1% (m/v) Casein Hydrolysate.

After 16 hours of incubation in high vacuum, electrodes were transferred to a desiccator to equilibrate for 1 hour. Electrode surfaces were submerged in a 1% (m/v) casein hydrolysate blocking buffer at pH 7.00 for 16 hours. Electrodes were removed, rinsed in deionised water by submersion, dried and stored in a desiccator for at least 1 hour prior to use.

2.5 Preparation of the Serum Dilutions with Rheumatoid Factor Blocker (RfB)

Serum is diluted to minimize the prozone or hook effect in samples with antibodies. Multiple serum concentrations can be used to control for the prozone effect. In this example, 1 in 1000 and 1 in 200 are used. As one example, a disposable well cell (Metrohm Dropsens, Spain) was attached to a coated, blocked electrode, a volume of 150 μl of 1% casein hydrolysate in 1 mM hexacyanoferrate buffer was pipetted into the well and the electrode was characterized using EIS as previously described. Thereafter serum was thawed and mixed with rheumatoid factor blocker (RfB) to a final concentration of 1 mg/ml and casein hydrolysate at 1% (w/v), and incubated for 15 minutes at 30° C. The serum was further diluted to 1 in 500 and 1 in 100 while maintaining the casein hydrolysate final concentrations at 1%. A volume of 75 μl was aspirated from the electrode, replaced with 75 μl of 1 in 500 dilution (to achieve 1 in 1000 effective concentration) of the serum/RfB sample solution in 1% casein hydrolysate in 1 mM hexacyanoferrate buffer, triturated to mix and incubated for 10 minutes. The electrode was then characterized a second time using EIS as previously described. A volume of 75 μl of the 1 in 1000 sample dilution was aspirated from the electrode, replaced with 75 μl of 1 in 100 sample dilution (1 in 200 effective concentration), triturated to mix and incubated for 10 minutes. The electrode was then characterized a third and last time using EIS as previously described.

### 3. Results

Minimum and maximum serum concentration are to be indicated by inclusion of the 1/200 dilution series of four unblinded sera. It is important to note that at 1/1600 dilution, these four serum samples gave no EIS signal above background. This enabled classification of the 1/1600 serum dilution as the lower bound concentration, with which not all TB-positive patient sera produce EIS signals higher than that obtained for TB-negative patient sera.

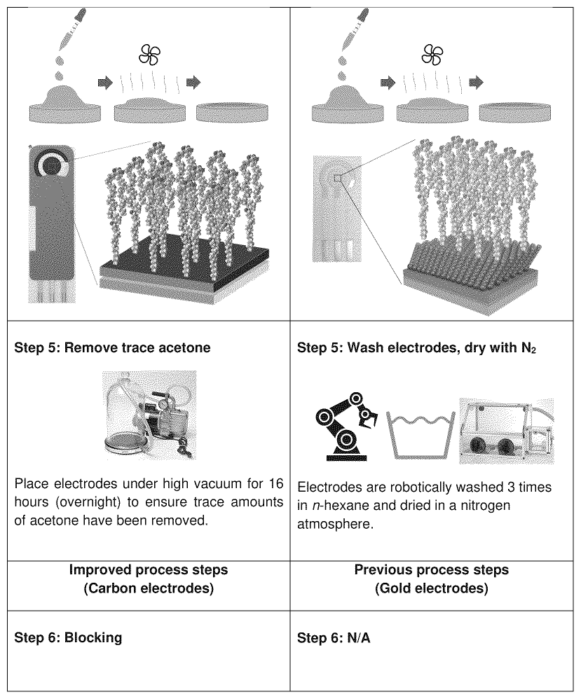

FIG. 11 demonstrates the impedance data before and after the acetone wash as well as after antigen immobilisation and blocking with casein hydrolysate.

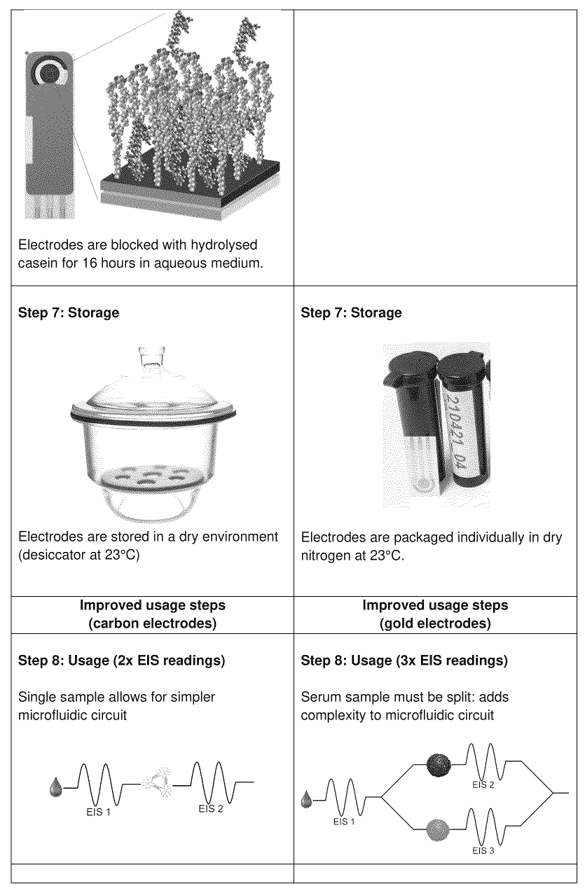

Using the acetone pre-treated carbon electrodes, the micellar MA coating process and surface blocking with casein hydrolysate, the MARTI-EIS technique is shown here to distinguish between two TB-positive and two TB-negative patient sera at both 1 in 1000 and 1 in 200 dilutions as a direct antibody binding assay. However, no distinction was possible for MVT112 and MVT 116 at the 1 in 1000 dilution. FIG. 12 demonstrates impedance data comparing TB+ and TB− sera at 1 in 1000 and 1 in 200 dilutions.

### 4. Discussion

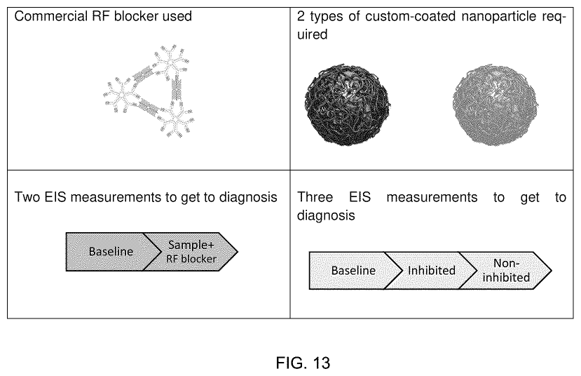

Assuming that the proven ability of the described method to distinguish two TB-positive from two TB-negative patients by analysis of their serum samples will apply to larger patient sample collections, the invention provides, amongst others, the following advantages of the prior art (EP2997371A1) as depicted in FIG. 13:

The carbon screen-printed electrodes for use in the invention are of consistent quality.

The tedious polishing of gold electrodes required previously, is now substituted with carbon electrodes requiring a simple, short chemical treatment with acetone. This was achieved by submerging the carbon electrodes in acetone for between 2 and 7 minutes, e.g. about 5 minutes. Previous use of gold electrodes required pre-cleaning using argon plasma and mechanical polishing with an alumina slurry.

Formation of an octadecanethiol (ODT) self-assembled monolayer is no longer needed as a preparatory step before coating with MA-antigen, thus shortening the process.

Use of gold electrodes and ODT required repeated washing in ethanol and drying in a nitrogen atmosphere overnight. In the current example, carbon electrodes merely require drying in an oven at 80° C. for between 10-30 minutes, e.g. about 20 minutes.

Immobilised MA from micellar acetone solution is presented in the desired, highly antigenic conformation for disease-specific antibody detection.

Upscaled automated MA antigen coating is enabled by robotic drop-deposition equipment. Acetone is generally compatible with such equipment and incompatible with the known solvents for molecular dissolution of MA, e.g., hexane and chloroform.

After coating, trace acetone is removed from the carbon electrodes by placing the electrodes under high vacuum. In comparison, previously the gold electrodes were washed 3 times in n-hexane and dried in a nitrogen atmosphere.

Unlike with the previous method which did not use blocking, the carbon electrodes of the present example are blocked with aqueous, hydrolysed casein for 16 hours.

The prepared carbon electrodes require simple storage in a dry environment, such as a desiccator at 23° C. With the previous method, the electrodes needed to be individually packed in dry nitrogen at 23° C.

With the previous method, the serum sample had to be split into two samples which required a more complex liquid processing. In the current invention requires three sequential impedance measurements to obtain the outcome of the test, thus allowing for a simplified design of a microfluidic circuit.

The previous state-of-the-art required two types of custom-coated nanoparticles. The current invention obviates the need for immunosorbent nanoparticle treatment of samples by replacement with much simpler addition of a rheumatoid factor blocker solution.

Table 2 below summarises the advantages of the present carbon electrode method in comparison to the previous gold electrode method.

TABLE 2
Advantages of the novel carbon electrode coating method

| | | |
|---|---|---|
| Parameter | Improved method | Previous method |

Electrode coating

| | | |
|---|---|---|
| Conductive material | Carbon | Gold |
| Electrode pre-treatment | Acetone soak | Plasma & |
| | | mechanical |
| | | polishing |
| Carrier solvent | Acetone | n-Hexane |
| Coating method | Drop-deposition | Immersion |
| Drying method | Dried in air at 80° C. | Dried in N₂ gas |
| | | within a |
| | | glove-box at 25° C. |
| Drying time | 20 minutes | Overnight |
| Number of coating steps | 2 | 3 |
| Post-coating processing | Vacuum to | Vacuum to |
| | remove trace | remove trace |
| | amounts | amounts of |
| | of acetone | n-hexane |
| Blocking | Casein hydrolysate | Not applicable |

EIS analysis

| | | |
|---|---|---|
| Favourable antigenicity | High | Low |
| of MA | | |
| Requirement for | No | Yes |
| nanoparticles | | |
| Use of rheumatoid | Yes | No |
| factor blocker | | |
| Non-specific binding | Minimal | High |

### Example 5

### Investigation of Chemical Blocking Agents

Most immunoassays require the use of a surface blocking agent to prevent adsorption of non-specific proteins and antibodies that would otherwise decrease the accuracy of the assay. During the early development of the MARTI EIS technique, typical blocking agents proved detrimental, causing impedance data to fall outside the detectable range. Due to differences in the manufacturing process, the change in electrode material from gold to carbon resulted in an increase in surface area, increasing adsorption of non-specific proteins and antibodies. Non-specific adsorption lowers the accuracy of diagnostic tests.

Approximately 40% of patient samples contain non-analyte proteins or antibodies. These interfering proteins can have low affinity and be present in high concentrations, or they can have high affinity and be present in low concentrations. These interfering proteins also vary from among patients (18).

Suppressing non-specific binding of serum enhances the signal-to-noise ratio of antibody binding. An ideal chemical blocking agent must be able to fill all the remaining sites after adsorption of antigen onto the surface (18). Typically, whole proteins are used as chemical blocking agents such as Bovine serum albumin (BSA) and casein. In this example, the efficacy of polyvinylpyrrolidone (PVP), a water-soluble polymer, and casein hydrolysate, a bacterial culture media supplement as blocking agents were compared.

### 1. Methods

Coating of Carbon SPEs Using 0.1 mg/ml MA-Acetone with Accelerated Airflow

A disposable pipette tip was used to carefully dispense 4 μl of a 0.1 mg/ml MA in acetone solution at between 25° C. and 35° C., in this case at 30° C., onto the working electrode of each SPE in 10 repeated deposition actions with accelerated airflow of 15 km/h. Electrodes were incubated under high vacuum≤50 mTorr for 16 hours≤50 mTorr.

1.2 Blocking of Carbon SPEs with 0.01% PVP and Casein Hydrolysate

Electrodes were transferred to a desiccator to equilibrate for 1 hour. Some electrode surfaces were submerged in a 0.01% (m/v) casein hydrolysate blocking buffer at pH 7.00 and some in a 0.01% (m/v) PVP blocking buffer at pH 7.00 for 16 hours. Electrodes were removed, rinsed in deionised water by submersion, dried and stored in a desiccator for at least 1 hour prior to use.

1.3 Comparison of 0.01% (m/v) PVP and Casein Hydrolysate Blocking Agent

A volume of 150 μl of 1 mM hexacyanoferrate buffer was pipetted onto the electrode, and the electrode was characterized using CV and EIS. The CV scans were cycled from −0.2 V to +0.4 V, with a voltage step of 2.44 mV at a scan rate of 50 mV/s. EIS scan occurred at a dc voltage of 0.135 V, AC amplitude of 0.01 V at 50 frequencies from 2000 to 0.1 Hz. Thereafter, serum was thawed and diluted with 1 mM hexacyanoferrate buffer to a concentration of 1 in 1600. The 1 mM hexacyanoferrate buffer on the electrode was aspirated and replaced by 150 μl of 1 in 1600 serum sample in 1 mM hexacyanoferrate and incubated for 10 minutes. The electrode was then characterized again using EIS as previously described.

### 2. Results

With PVP, impedance data was out of range, even when titrated as low as 0.01%. No distinction between TB-positive and TB-negative sera was obtained using PVP as a surface blocking agent, providing evidence that PVP was incompatible with this system. However, casein hydrolysate at 0.1%, successfully distinguished between TB-positive and TB-negative sera.

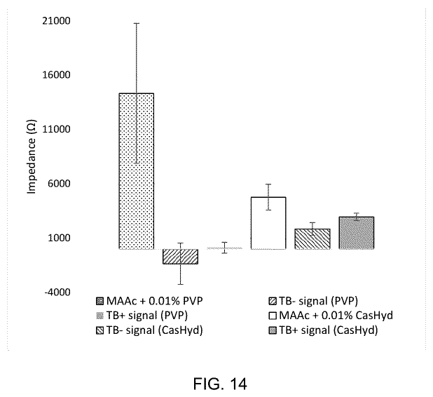

FIG. 14 demonstrates that PVP blocking significantly increases the impedance signals of the MA coating, reducing the window of detection such that no statistically significant difference is seen between the TB-positive and TB-negative serum samples. Conversely, casein hydrolysate blocking improves antibody detection, and a difference in impedance values is attained between TB-positive and TB-negative serum samples.

### Example 6

### Rheumatoid Factor Blocker as Serum Additive to Improve Accuracy

Elkayam et al. (19) showed that high levels of rheumatoid factor are present in most TB patients in the active disease state. Rheumatoid factors bind to the non-specific, fragment crystallizable (Fc) regions of antibodies, thereby forming complexes that effectively minimise the number of TB antigen specific antibodies available for detection. Immunoassays are confounded by interfering antibodies like autoantibodies that alter the measurable concentration of specific antibodies to the antigen. Consequently, data may be misinterpreted due to false specific-antibody binding concentrations (20).

In this example, a rheumatoid factor (RF) interference suppressor, Rheumatoid factor interference blocker, consisting of a proprietary, highly concentrated, blend of anti-RF antibodies is used to reduce interference and improve antibody binding accuracy.

### 1.1 Methods

1.1 Coating of Carbon SPEs Using 0.1 mg/ml MA-Acetone with Accelerated Airflow

A plastic pipette was used to carefully dispense 4 μl of a 0.1 mg/ml MA in acetone solution between 25° C. and 35° C., in this case at 30° C., onto the working electrode of each SPE in 10 repeated deposition actions with accelerated airflow of 15 km/h. Electrodes were incubated under high vacuum <=50 mTorr for 16 hours <=50 mTorr.

1.2 Blocking of Carbon SPEs with 1% (m/v) Casein Hydrolysate

Electrodes were transferred to a desiccator to equilibrate for 1 hour. Electrode surfaces were submerged in a 1% (m/v) casein hydrolysate blocking buffer at pH 7.00 for 16 hours. Electrodes were removed, rinsed in deionised water by submersion, dried and stored in a desiccator for at least 1 hour prior to use.

1.3 Reducing Interference with Rheumatoid Factor Blocker (RfB)

A volume of 150 μl of 1 mM hexacyanoferrate buffer was pipetted onto the electrode, and the electrode was characterized using CV and EIS. The CV scans were cycled from −0.2 V to +0.4 V, with a voltage step of 2.44 mV at a scan rate of 50 mV/s. EIS scan occurred at a dc voltage of 0.135 V, A amplitude of 0.01 V at 50 frequencies from 2000 to 0.1 Hz. Thereafter, the serum was thawed and diluted with 1 mM hexacyanoferrate buffer only, or 1 mM hexacyanoferrate buffer and RfB (1 mg/ml) to a concentration of 1 in 1600. The 1 mM hexacyanoferrate buffer on the electrode was aspirated and replaced on one electrode by 150 μl of 1 in 1600 serum sample in 1 mM hexacyanoferrate, on the second electrode by 150 μl of 1 in 1600 serum sample and 1 mg/ml RfB in 1 mM hexacyanoferrate and both electrodes subsequently incubated for 10 minutes. Impedance signals were then generated using the EIS technique as previously described.

### 2. Results

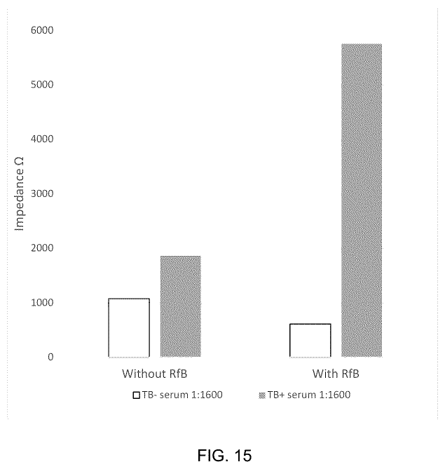

Using the same serum sample set as was used in Example 5, each sample incubated either with or without RfB, impedance signals were generated to detect the binding of anti-mycolic acid antibodies. FIG. 15 demonstrates that RfB enhances the impedance signals of antibody binding and therefore improves the quality of distinction between TB-positive patient and TB-negative control sera.

### 3. Conclusion

By blocking the activity of rheumatoid factor, non-specific antibody binding is impaired and specific anti-mycolic acid antibody is enhanced. With more than half of TB patients affected by rheumatoid factor in the circulation (19), it is proved that it is important to use rheumatoid factor blocker in serum samples to improve the outcome of the test to detect active TB surrogate marker antibodies to mycolic acid.

In early versions of the MARTI assay, nanoparticles proved an essential immunosorbent to minimise the effects of cross-reacting anti-cholesterol antibodies and to amplify the difference in signal between inhibited and non-inhibited TB patient samples. Our data (Example 4, Table 1) suggests that the PLGA nanoparticle immunosorbent is no longer needed to ensure accuracy of the MARTI diagnostic test. Rheumatoid factor blocker has effectively obviated the need for the nanoparticles by minimising the effect of antibody cross-linking rheumatoid factor.

This discovery simplifies the diagnostic procedure, by excluding an inhibition step that requires splitting of the sample in two parts, each requiring EIS measurements per sample. Direct antibody binding in a single sample exposure to the electrode is now adequate, while providing a sufficient signal window to distinguish between TB-positive and TB-negative patient sera.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method that “comprises,” “has,” “includes,” or “contains” one or more steps or elements possesses those one or more steps or elements, but is not limited to possessing only those one or more steps or elements. Likewise, a step of a method that “comprises,” “has,” “includes,” or “contains” one or more features possesses those one or more features, but is not limited to possessing only those one or more features. Furthermore, a method or device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. A method of forming a micellar solution of mycolic acid antigens for immobilisation on an electrode, the method comprising heating a mixture of mycolic acid and acetone to a temperature higher than the melting point of the mycolic acid, thus producing a solution of mycolic acid antigens in acetone, and cooling the solution to produce a micellar solution of mycolic acid antigens in acetone.

2. The method according to claim 1, wherein the temperature higher than the melting point of mycolic acid is a temperature between 60° C. and 90° C.

3. The method according to claim 1 or claim 2, wherein the solution of mycolic acid antigens in acetone is cooled to a temperature between 25° C. and 35° C.

4. The method according to any one of claims 1 to 3, which includes a prior step of forming the mixture of mycolic acid and acetone to a concentration of between 0.05 and 0.25 mg/ml by adding the mycolic acid to the acetone.

5. A micellar solution of mycolic acid antigens in acetone produced according to the method according to any one of claims 1 to 4.

6. A method of immobilising mycolic acid antigens on an electrode, the method including applying the micellar solution of mycolic acid antigens in acetone according to claim 5 to an electrode on which mycolic acid antigens are to be immobilised.

7. The method according to claim 6, which includes a prior step of producing the micellar solution of mycolic acid antigens in acetone according to the method of any one of claims 1 to 4.

8. The method according to claim 7, wherein the step of applying the micellar solution of mycolic acid antigens in acetone to the electrode is performed within four hours of the heating of a mixture of mycolic acid and acetone according to the method of any one of claims 1 to 4.

9. The method according to any one of claims 6 to 8, which includes leaving or causing the micellar solution of mycolic acid antigens in acetone to dry on the electrode.

10. The method according to any one of claims 6 to 9, wherein the electrode is free of a surface modifying monolayer.

11. The method according to any one of claims 6 to 10, which includes a subsequent step of blocking non-specific binding sites on the solid surface by treating the electrode with a protein hydrolysate.

12. The method according to claim 11, wherein the protein hydrolysate is provided as a solution of casein hydrolysate.

13. The method according to any one of claims 6 to 12, wherein the electrode is a screen-printed carbon electrode.

14. The method according to claim 13, wherein the screen-printed carbon electrode is a screen-printed carbon electrode treated with acetone to remove organic binders and impurities from the electrode, for immobilising mycolic acid antigens thereon in the absence of a surface modifying monolayer.

15. The method according to claim 14, which includes a prior step of preparing the screen-printed carbon electrode by contacting a screen-printed carbon electrode with acetone to remove organic binders and impurities from the electrode.

16. The method according to claim 15, wherein contacting the screen-printed carbon electrode with acetone includes submerging the electrode in, or spraying or flowing the electrode with acetone; and/or
rinsing the solid surface with acetone,
wherein, if both submerging and rinsing are performed, rinsing is performed after submerging.

17. The method according to claim 15 or claim 16, which includes drying the electrode after treating the electrode with acetone.

18. The method according to any one of claims 15 to 17, subject to the proviso that the method omits a step of applying a surface modifying monolayer to the electrode.

19. A screen-printed carbon electrode comprising mycolic acid antigens immobilised thereon, produced according to the method according to any one of claims 13 to 18.

20. A diagnostic kit for diagnosing tuberculosis in a human or animal subject by electro-impedance spectroscopy, the kit comprising a screen-printed carbon electrode according to claim 19.

21. A method of detecting tuberculosis biomarker antibodies in a human or animal blood or tissue sample, the method including contacting an electrode on which mycolic acid antigens have been immobilised according to the method of any one of claims 6 to 18, or an electrode according to claim 19, with a sample from a patient suspected of having active tuberculosis in order to allow any biomarker anti-mycolic acid antibodies in the sample to bind to the immobilised mycolic acid antigens.

22. A method of immobilising mycolic acid antigens on an electrode, for detecting tuberculosis biomarker antibodies in a human or animal blood or tissue sample, the method including
forming a micellar solution of mycolic acid in acetone by forming a mixture of mycolic acid and acetone, heating the mixture to between 60° C. and 90° C., and thereafter cooling the mixture to between 25° C. and 35° C.;
contacting the micellar solution of mycolic acid in acetone with an electrode; and
leaving or causing the micellar solution of mycolic acid in acetone to dry on the electrode.

23. The method according to claim 22, wherein the electrode is free of a surface-modifying monolayer.

24. The method according to claim 22 or claim 23, wherein the electrode is a screen-printed carbon electrode.
